(19) United States
(12) Patent Application Publication
(10) Pub. No.: US 2024/0417485 A1
(43) Pub. Date: Dec. 19, 2024

(54) uPAR Binding Compositions and Uses Therefor

(71) Applicant: University of Pittsburgh - Of the Commonwealth System of Higher Education, Pittsburgh, PA (US)

(72) Inventors: Dimiter Stanchev Dimitrov, St. Augustine, FL (US); John W. Mellors, Pittsburgh, PA (US); Wei Li, Pittsburgh, PA (US); Xiaojie Chu, Pittsburgh, PA (US)

(21) Appl. No.: 18/743,923

(22) Filed: Jun. 14, 2024

Related U.S. Application Data
(60) Provisional application No. 63/508,550, filed on Jun. 16, 2023.

(51) Int. Cl. C07K 16/28; A61K 47/68; A61P 35/00

(52) U.S. Cl. CPC C07K 16/2896; A61K 47/6803; A61K 47/68031; A61K 47/68033; A61K 47/68035; A61K 47/6809; A61K 47/6831; A61K 47/6851; A61K 47/6889; A61P 35/00; C07K 2317/31; C07K 2317/92

(57) ABSTRACT

Provided herein are single-domain antigen binding molecules able to bind uPAR. Also provided herein are methods of treating cancer, fibrotic disease, or diseases related to senescent cell phenotypes in which uPAR is expressed.

### CROSS REFERENCE TO RELATED APPLICATIONS

This application claims priority to U.S. Provisional Patent Application No. 63/508,550 filed Jun. 16, 2023, the disclosure of which is incorporated herein by reference in its entirety.

### REFERENCE TO A SEQUENCE LISTING

The Sequence Listing associated with this application is filed in electronic format and is hereby incorporated by reference into the specification in its entirety. The name of the XML file containing the Sequence Listing is 2401878.xml. The size of the XML file is 27,061 bytes and the XML file was created on Jun. 11, 2024.

The urokinase-type plasminogen activator receptor (uPAR, also named CD87) is the cell surface receptor for the extracellular serine protease urokinase-type plasminogen activator (uPA, or urokinase). uPAR both regulates the proteolysis of the extracellular matrix (ECM) by engaging uPA for activation of the plasminogen activation system and initiates signaling involved in cell adhesion, migration, proliferation, and survival by interacting with other partners, such as integrins. uPAR is highly expressed in various human cancers and that it plays multiple roles in the development of cancer. uPAR is a cysteine-rich glycosylated protein with three domains connected by short linker regions namely D1, D2, and D3. Transmembrane glycerophosphodiesterase GDE3 can cleave the GPI anchor and release uPAR from the cell membrane surface to produce the soluble uPAR. In recent years, various studies have shown that uPAR is overexpressed in many solid cancers such as breast cancer, prostate cancer, bladder cancer, and colorectal cancer. The high expression level of uPAR also has a lower survival rate and poor prognosis. And it is also associated with tumor cell proliferation, cell adhesion, metastasis, glycolysis, and tumor angiogenesis. Thus, uPAR has emerged as an important target in cancers (See, e.g., Wang L, Lin X, Sun P. uPAR, beyond regulating physiological functions, has orchestrated roles in cancer (Review). Int J Oncol. 2022 December; 61 (6): 151). Moreover, recent reports have shown that uPAR is broadly induced during cell senescence and targeting uPAR could efficiently decrease senescent cells in senescence-associated disease (See, e.g., Amor C, Feucht J, Leibold J, Ho Y J, Zhu C, Alonso-Curbelo D, Mansilla-Soto J, Boyer J A, Li X, Giavridis T, Kulick A, Houlihan S, Peerschke E, Friedman S L, Ponomarev V, Piersigilli A, Sadelain M, Lowe S W. Senolytic CAR T cells reverse senescence-associated pathologies. Nature. 2020 July; 583 (7814): 127-132).

Senolescence, characterized by stable cell-cycle arrest and a secretory program that modulates the tissue microenvironment, is a tumor defense mechanism that prevents expansion of malignancies. Consequently, therapies that target growing malignant cell populations often do not target senolescent cells, which can later contribute to malignancies and fibrosis. Senolescence also contributes to diseases such as liver and lung fibrosis, atherosclerosis, diabetes, and osteoarthritis. As such, the ability to target senolescent cells is desirable. Amor et al. described CAR T therapies targeting uPAR useful for eliminating senescent cells.

Qin et al. (Qin L, Wang L, Zhang J, Zhou H, Yang Z, Wang Y, Cai W, Wen F, Jiang X, Zhang T, Ye H, Long B, Qin J, Shi W, Guan X, Yu Z, Yang J, Wang Q, Jiao Z. Therapeutic strategies targeting uPAR potentiate anti-PD-1 efficacy in diffuse-type gastric cancer. Sci Adv. 2022 May 27; 8 (21): eabn3774) provide evidence in support of targeting uPAR as an adjuvant therapy to anti-PD-1 (anti-Programmed cell death protein 1) in treatment of diffuse-type gastric cancer. This supports an adjuvant therapy using anti-uPAR antibody reagents in combination with anti-PD-1 therapies, such as nivolumab or pembrolizumab, or in combination with anti-Human epidermal growth factor receptor 2 (HER2) therapies, such as antibody (e.g., trastuzumab) therapies.

Kanno Y. (The uPA/uPAR System Orchestrates the Inflammatory Response, Vascular Homeostasis, and Immune System in Fibrosis Progression. Int J Mol Sci. 2023 Jan. 16; 24 (2): 1796) describes the significant role uPAR plays in fibrotic disease, for example in systemic sclerosis (SSc), idiopathic pulmonary fibrosis, renal fibrosis, and liver cirrhosis.

Identification of binding reagents, comprising binding domains (e.g., paratopes) targeting uPAR with high affinity, and avidity. Such binding domains find use in antibody therapies and in related therapies, such as CAR T therapies.

### SUMMARY

An antigen binding molecule, such as a single domain antibody or a bi-specific T cell engager, is provided comprising a polypeptide comprising a uPAR-binding amino acid sequence, an antigen binding site, or CDRs of:

(SEQ ID NO: 1)
EVQLVESGGGLVQPGGSLRLSCAASGFTFSRYWMSWVRQAPGKALEWIGEI
NHSGSTNYNPSLKSLVTISRDNSKNTLYLQMNSLRAEDTATYYCARSLVPA
LSYYYYYGMDVWGQGTTVTVSS;
(SEQ ID NO: 3)
EVQLVESGGGLVQPGGSLRLSCKGSGFTFGDYAIGWVRQAPGQRLEWIGWI
NTNSGSPKYAQGFTGRFTISRDNSKNTLYLQMNSLRAEDTAVYYCATDVVV
PWGQGSQVTVSS,

or a sequence having at least 70%, at least 75%, at least 80%, at least 90%, or at least 95% sequence identity with either of the preceding. A composition is provided comprising the antigen binding molecule and a pharmaceutically-acceptable excipient. An antibody drug conjugate (ADC) is provided comprising an antibody binding molecule linked to a cytotoxic payload via a chemical linker. A composition comprising the ADC and a pharmaceutically-acceptable excipient also is provided. Where the antigen binding molecule is a chimeric antigen receptor, a cell, such as a CAR T or CAR NK cell is provided, comprising the antigen-binding molecule.

An antigen binding reagent is provided comprising a means for binding a uPAR protein on a surface of a cell comprising CDRs of VH3 or VH115.

A method of reducing a population of uPAR-expressing cells in a patient, is provided, comprising administering to the patient an amount of a composition or cell as described in the preceding paragraph effective to reduce the number of cells of the population of uPAR-expressing cells in the patient. The method may be used for treating cancer or a fibrotic disease.

The following numbered clauses outline various illustrative embodiments, aspects, and examples of the present invention.

Clause 1. An antigen binding molecule comprising a polypeptide comprising a uPAR-binding amino acid sequence, an antigen binding site, or CDRs of:

(SEQ ID NO: 1)
EVQLVESGGGLVQPGGSLRLSCAASGFTFSRYWMSWVRQAPGKALEWIGEI
NHSGSTNYNPSLKSLVTISRDNSKNTLYLQMNSLRAEDTATYYCARSLVPA
LSYYYYYGMDVWGQGTTVTVSS;
(SEQ ID NO: 3)
EVQLVESGGGLVQPGGSLRLSCKGSGFTFGDYAIGWVRQAPGQRLEWIGWI
NTNSGSPKYAQGFTGRFTISRDNSKNTLYLQMNSLRAEDTAVYYCATDVVV
PWGQGSQVTVSS,

or a sequence having at least 70%, at least 75%, at least 80%, at least 90%, or at least 95% sequence identity with either of the preceding.

Clause 2. The antigen binding molecule of clause 1, comprising a uPAR-binding amino acid sequence having at least 70%, at least 75%, at least 80%, at least 90%, at least 95% or 100% sequence identity with:

(SEQ ID NO: 1)
EVQLVESGGGLVQPGGSLRLSCAASGFTFSRYWMSWVRQAPGKALEWIGEI
NHSGSTNYNPSLKSLVTISRDNSKNTLYLQMNSLRAEDTATYYCARSLVPA
LSYYYYYGMDVWGQGTTVTVSS.

Clause 3. The antigen binding molecule of clause 1, wherein the uPAR-binding amino acid sequence comprises the amino acid sequences:

(SEQ ID NO: 13)
VH3-HCDR1: RYWMS;
(SEQ ID NO: 14)
VH3-HCDR2: EINHSGSTNYNPSLKS;
and
(SEQ ID NO: 15)
VH3-HCDR3: SLVPALSYYYYYGMDV.

Clause 4. The antigen binding molecule of clause 1, comprising a uPAR-binding amino acid sequence having at least 70%, at least 75%, at least 80%, at least 90%, at least 95% or 100% sequence identity with:

(SEQ ID NO: 3)
EVQLVESGGGLVQPGGSLRLSCKGSGFTFGDYAIGWVRQAPGQRLEWIGW
INTNSGSPKYAQGFTGRFTISRDNSKNTLYLQMNSLRAEDTAVYYCATDV
VVPWGQGSQVTVSS.

Clause 5. The antigen binding molecule of clause 1, wherein the uPAR-binding amino acid sequence comprises the amino acid sequences:

(SEQ ID NO: 16)
VH115-HCDR1: DYAIG;
(SEQ ID NO: 17)
VH115-HCDR2: WINTNSGSPKYAQGFTG;
and
(SEQ ID NO: 18)
VH115-HCDR3: DVVVP.

Clause 6. The antigen binding molecule of any one of clauses 1-5, in the form of a nanobody comprising the uPAR-binding amino acid sequence.

Clause 7. The antigen binding molecule of clause 6, having a sequence:

(SEQ ID NO: 1)
EVQLVESGGGLVQPGGSLRLSCAASGFTFSRYWMSWVRQAPGKALEWIGE
INHSGSTNYNPSLKSLVTISRDNSKNTLYLQMNSLRAEDTATYYCARSLV
PALSYYYYYGMDVWGQGTTVTVSS,
or

having at least 70%, at least 75%, at least 80%, at least 90%, at least 95%, or 100% sequence identity with:

(SEQ ID NO: 1)
EVQLVESGGGLVQPGGSLRLSCAASGFTFSRYWMSWVRQAPGKALEWIGE
INHSGSTNYNPSLKSLVTISRDNSKNTLYLQMNSLRAEDTATYYCARSLV
PALSYYYYYGMDVWGQGTTVTVSS.

Clause 8. The antigen binding molecule of clause 6, having a sequence:

(SEQ ID NO: 3)
EVQLVESGGGLVQPGGSLRLSCKGSGFTFGDYAIGWVRQAPGQRLEWIGW
INTNSGSPKYAQGFTGRFTISRDNSKNTLYLQMNSLRAEDTAVYYCATDV
VVPWGQGSQVTVSS;
or

having at least 70%, at least 75%, at least 80%, at least 90%, at least 95%, or 100% sequence identity with:

(SEQ ID NO: 3)
EVQLVESGGGLVQPGGSLRLSCKGSGFTFGDYAIGWVRQAPGQRLEWIG
WINTNSGSPKYAQGFTGRFTISRDNSKNTLYLQMNSLRAEDTAVYYCAT
DVVVPWGQGSQVTVSS.

Clause 9. The antigen binding molecule of any one of clauses 1-5, in the form of a V_{H}-Fc fusion protein, comprising the uPAR-binding amino acid sequence.

Clause 10. The antigen binding molecule of clause 9, having a sequence:

(SEQ ID NO: 5)
EVQLVESGGGLVQPGGSLRLSCAASGFTFSRYWMSWVRQAPGKALEWIG
EINHSGSTNYNPSLKSLVTISRDNSKNTLYLQMNSLRAEDTATYYCARS
LVPALSYYYYYGMDVWGQGTTVTVSSSGDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK,
or

having at least 70%, at least 75%, at least 80%, at least 90%, at least 95%, or 100% sequence identity with:

(SEQ ID NO: 5)
EVQLVESGGGLVQPGGSLRLSCAASGFTFSRYWMSWVRQAPGKALEWIG
EINHSGSTNYNPSLKSLVTISRDNSKNTLYLQMNSLRAEDTATYYCARS
LVPALSYYYYYGMDVWGQGTTVTVSSSGDKTHTCPPCPAPELLGGPSVF
LFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYT
QKSLSLSPGK.

Clause 11. The antigen binding molecule of clause 9, having a sequence:

(SEQ ID NO: 7)
EVQLVESGGGLVQPGGSLRLSCKGSGFTFGDYAIGWVRQAPGQRLEWIG
WINTNSGSPKYAQGFTGRFTISRDNSKNTLYLQMNSLRAEDTAVYYCAT
DVVVPWGQGSQVTVSSSGDKTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
K;
or

having at least 70%, at least 75%, at least 80%, at least 90%, at least 95%, or 100% sequence identity with:

(SEQ ID NO: 7)
EVQLVESGGGLVQPGGSLRLSCKGSGFTFGDYAIGWVRQAPGQRLEWIG
WINTNSGSPKYAQGFTGRFTISRDNSKNTLYLQMNSLRAEDTAVYYCAT
DVVVPWGQGSQVTVSSSGDKTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPG
K.

Clause 12. The antigen binding molecule of any one of clauses 1-5, in the form of a bi-specific T-cell engager, comprising the uPAR-binding amino acid sequence.

Clause 13. The antigen binding molecule of clause 12, having a sequence:

(SEQ ID NO: 9)
EVQLVESGGGLVQPGGSLRLSCAASGFTFSRYWMSWVRQAPGKALEWIG
EINHSGSTNYNPSLKSLVTISRDNSKNTLYLQMNSLRAEDTATYYCARS
LVPALSYYYYYGMDVWGQGTTVTVSSGSGSSGSGSSGSGSSQVQLVQSG
GGVVQPGRSLRLSCKASGYTFTRYTMHWVRQAPGKGLEWIGYINPSRGY
TNYNQKVKDRFTISRDNSKNTAFLQMDSLRPEDTGVYFCARYYDDHYCL
DYWGQGTPVTVSSGGGGSGGGGSGGGGSGGGGSGGGGSGGGGSDIQMTQ
SPSSLSASVGDRVTITCSASSSVSYMNWYQQTPGKAPKRWIYDTSKLAS
GVPSRFSGSGSGTDYTFTISSLQPEDIATYYCQQWSSNPFTFGQGTKLQ
ITRGSGSSGDKTHTCPPCPAPEAAGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALGAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGKGSHHHHHH,
or

having at least 70%, at least 75%, at least 80%, at least 90%, at least 95% or 100% sequence identity with:

(SEQ ID NO: 9)
EVQLVESGGGLVQPGGSLRLSCAASGFTFSRYWMSWVRQAPGKALEWIG
EINHSGSTNYNPSLKSLVTISRDNSKNTLYLQMNSLRAEDTATYYCARS
LVPALSYYYYYGMDVWGQGTTVTVSSGSGSSGSGSSGSGSSQVQLVQSG
GGVVQPGRSLRLSCKASGYTFTRYTMHWVRQAPGKGLEWIGYINPSRGY
TNYNQKVKDRFTISRDNSKNTAFLQMDSLRPEDTGVYFCARYYDDHYCL
DYWGQGTPVTVSSGGGGSGGGGSGGGGSGGGGSGGGGSGGGGSDIQMTQ
SPSSLSASVGDRVTITCSASSSVSYMNWYQQTPGKAPKRWIYDTSKLAS
GVPSRFSGSGSGTDYTFTISSLQPEDIATYYCQQWSSNPFTFGQGTKLQ
ITRGSGSSGDKTHTCPPCPAPEAAGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALGAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGKGSHHHHHH.

Clause 14. The antigen binding molecule of clause 12, having a sequence:

(SEQ ID NO: 11)
EVQLVESGGGLVQPGGSLRLSCKGSGFTFGDYAIGWVRQAPGQRLEWIG
WINTNSGSPKYAQGFTGRFTISRDNSKNTLYLQMNSLRAEDTAVYYCAT
DVVVPWGQGSQVTVSSGSGSSGSGSSGSGSSQVQLVQSGGGVVQPGRSL
RLSCKASGYTFTRYTMHWVRQAPGKGLEWIGYINPSRGYTNYNQKVKDR
FTISRDNSKNTAFLQMDSLRPEDTGVYFCARYYDDHYCLDYWGQGTPVT
VSSGGGGSGGGGSGGGGSGGGGSGGGGSGGGGSDIQMTQSPSSLSASVG
DRVTITCSASSSVSYMNWYQQTPGKAPKRWIYDTSKLASGVPSRFSGSG
SGTDYTFTISSLQPEDIATYYCQQWSSNPFTFGQGTKLQITRGSGSSGD
KTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALGAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPGKGSHHHHHH;
or

having at least 70%, at least 75%, at least 80%, at least 90%, at least 95% or 100% sequence identity with:

(SEQ ID NO: 11)
EVQLVESGGGLVQPGGSLRLSCKGSGFTFGDYAIGWVRQAPGQRLEWIG
WINTNSGSPKYAQGFTGRFTISRDNSKNTLYLQMNSLRAEDTAVYYCAT
DVVVPWGQGSQVTVSSGSGSSGSGSSGSGSSQVQLVQSGGGVVQPGRSL
RLSCKASGYTFTRYTMHWVRQAPGKGLEWIGYINPSRGYTNYNQKVKDR
FTISRDNSKNTAFLQMDSLRPEDTGVYFCARYYDDHYCLDYWGQGTPVT
VSSGGGGSGGGGSGGGGSGGGGSGGGGSGGGGSDIQMTQSPSSLSASVG
DRVTITCSASSSVSYMNWYQQTPGKAPKRWIYDTSKLASGVPSRFSGSG
SGTDYTFTISSLQPEDIATYYCQQWSSNPFTFGQGTKLQITRGSGSSGD
KTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALGAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPGKGSHHHHHH.

Clause 15. The antigen binding molecule of any one of clauses 1-15, wherein the K_{D} for antigen binding of the uPAR-binding amino acid sequence to uPAR protein is 100 nM or less, 75 nM or less, 50 nM or less, or 25 nM or less, as determined by surface plasmon resonance or biolayer interferometry (BLITZ).

Clause 16. A composition comprising the antigen binding molecule of any one of clauses 1-15 and a pharmaceutically-acceptable excipient.

Clause 17. The antigen binding molecule of any one of clauses 1-5 or 15, in the form of a chimeric antigen receptor, comprising the uPAR-binding amino acid sequence.

Clause 18. A T-cell or NK cell comprising the antigen binding molecule of clause 17.

Clause 19. An antibody drug conjugate (ADC) comprising an antibody binding molecule ligand as in any one of clauses 1-15 linked to a cytotoxic payload via a chemical linker.

Clause 20. The ADC of clause 19, wherein the cytotoxic payload is a microtubule-disrupting agent, a DNA-damaging agent, an RNA-targeting payload, an immune payload, a Bcl-xL inhibitor, a NAMPT inhibitor, or proteasome inhibitors.

Clause 21. The ADC of clause 19, wherein the cytotoxic payload is auristatin, a maytansinoid, an eribulin (e.g., eribulin mesylate), a tubulysins, a cryptophycins, an EG5 inhibitor, calicheamicin, a duocarmycin, doxorubicin, enediyne, a topoisomerase I inhibitor, a pyrrolo[2,1-c][1,4] benzodiazepine, a thailanstatins, an amatoxin, a Toll-like receptor agonist, a STING agonist, a glucocorticoid receptor modulator, or a carmaphycin.

Clause 22. The ADC of any one of clauses 19-21, wherein the chemical linker is an acid-labile linker, a lysosomal protease-sensitive linker, a 3-glucuronide linker, a glutathione-sensitive disulfide linker, such as an ester-containing linker, a hydrazone-containing linker, a valine-citrulline (v-c)-containing linker, a valine-alanine (v-a)-containing linker, or a phenylalanine-lysine (p-I)-containing linker.

Clause 23. A composition comprising the ADC of any one of clauses 19-22 and a pharmaceutically-acceptable excipient.

Clause 24. An antigen binding reagent comprising a means for binding a uPAR protein on a surface of a cell comprising CDRs of VH3 or VH115.

Clause 25. A method of reducing a population of uPAR-expressing cells in a patient, comprising administering to the patient an amount of the composition of clause 16 or 23 or the cell of clause 18 effective to reduce the number of cells of the population of uPAR-expressing cells in the patient.

Clause 26. A method of treating a cancer in a patient, comprising administering to the patient an amount of the composition of clause 16 or 23 or the cell of clause 18 effective to treat the cancer in the patient.

Clause 27. The method of clause 26, wherein the cancer is breast cancer, colorectal cancer, melanoma, brain cancer, lung cancer, ovarian cancer, prostate cancer, bladder cancer, liver cancer, gastric cancer, pancreatic cancer, a glioma, or a hematologic malignancy, such as acute myeloid leukemia or myeloma.

Clause 28. A method of treating a fibrotic disease in a patient, comprising administering to the patient an amount of the composition of clause 16 or 23 or the cell of clause 18 effective to treat the fibrotic disease in the patient.

Clause 27. The method of clause 26, wherein the fibrotic disease is systemic sclerosis (SSc), idiopathic pulmonary fibrosis, renal fibrosis, ureteral fibrosis, urethral fibrosis, bladder fibrosis, Peyronie's disease, liver cirrhosis, or COPD.

Clause 28. A method of treating a patient having a senescence-related disease, such as fibrin-associated inflammation or liver fibrosis, comprising administering to the patient an amount of the composition of clause 16 or 23 or the cell of claim 18 effective to treat the senescence-related disease in the patient.

### BRIEF DESCRIPTION OF THE DRAWINGS

FIGS. 1A-1D. Specificity of V_{H}/V_{H}-Fc with human uPAR on cell surface: (FIG. 1A) Anti-uPAR V_{H} and V_{H}-Fc binding to recombinant human uPAR measured by ELISA (left), and competition BLItz of V_{H} 3 with V_{H} 115 at 500 nM for binding to uPAR (right); (FIG. 1B-1D) Cell surface detection of uPAR by commercial mouse anti-uPAR antibody, V_{H} 3 and 115 (1 uM), and V_{H}-Fc 3 and 115 (50 nM) on 293T, 293T-uPAR, and A375 cells; (FIG. 1E) Dose-dependent cell surface binding of V_{H}-Fc 3 and 115 on 293T-uPAR cells. (FIG. 1F) Aggregation evaluation of V_{H} 3 and V_{H}-Fc 3 (left), V_{H} 115 and V_{H}-Fc 115 (right) measured by SEC.

FIGS. 2A-2F. In vitro cytotoxicity of T cells to uPAR-expressing cells by anti-uPAR DbTE: (FIGS. 2A and 2B) DbTE 3 and 115 binding to uPAR (FIG. 2A) and CD3 (FIG. 2B) measured by ELISA. (FIG. 2C) Cell binding of DbTE 3 and 115 on T cells tested by flow cytometry. (FIGS. 2D-2F) Percent relative lysis of 293T cells (FIG. 2D), 293T-uPAR cells (FIG. 2E), and A375 cells (FIG. 2F) by T cells mediated by DbTE 3 and 115, respectively. T cells and target cells were added at E: T ratio of 10:1 and simultaneously treated with serially diluted DbTE antibodies for 24 h. Experiment was repeated two times. Values were reported as the mean of percent relative lysis+SD. Significance was tested by using two-way ANOVA, followed by Tukey's multiple comparisons test. ****, p<0.0001.

FIGS. 3A and 3B provide amino acid sequences (top) and corresponding DNA sequences (bottom) for VH 3 (FIG. 3A, SEQ ID NOS: 1 and 2) and VH 115 (FIG. 3B, SEQ ID NOS: 3 and 4).

FIGS. 4A and 4B provide amino acid sequences (top) and corresponding DNA sequences (bottom) for VH-FC 3 (FIG. 4A, SEQ ID NOS: 5 and 6) and VH-Fc 115 (FIG. 4B, SEQ ID NOS: 7 and 8).

FIGS. 5A and 5B provide amino acid sequences (top) and corresponding DNA sequences (bottom) for DbTE 3 (FIG. 5A, SEQ ID NOS: 9 and 10) and DbTE 115 (FIG. 5B, SEQ ID NOS: 11 and. 12). Amino acid sequences are contiguous but are broken up into constituent sections by line breaks for illustration purposes only. For illustration purposes only, DNA sequences are presented in alternate upper-case sections and lower-case sections to indicate sequences encoding the different constituent sections depicted in the respective amino acid sequences.

### DETAILED DESCRIPTION

Other than in the operating examples, or where otherwise indicated, the use of numerical values in the various ranges specified in this application are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. Further, as used herein, all numbers expressing dimensions, physical characteristics, processing parameters, quantities of ingredients, reaction conditions, and the like, used in the specification and claims are to be understood as being modified in all instances by the term “about”. Moreover, unless otherwise specified, all ranges disclosed herein are to be understood to encompass the beginning and ending range values and any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 3.3, 4.7 to 7.5, 5.5 to 10, and the like.

As used herein “a” and “an” refer to one or more. The term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention.

As used herein, spatial, or directional terms, such as “left”, “right”, “inner”, “outer”, “above”, “below”, “over”, “under”, and the like, relate to the invention as it is shown in the drawing figures are provided solely for ease of description and illustration, and do not imply directionality, unless specifically required for operation of the described aspect of the invention. It is to be understood that the invention can assume various alternative orientations and, accordingly, such terms are not to be considered as limiting.

As used herein, a “patient” or “subject” is an animal, such as a mammal, including a primate (such as a human, a non-human primate, e.g., a monkey and a chimpanzee), a non-primate (such as a cow, a pig, a camel, a llama, a horse, a goat, a rabbit, a sheep, a hamster, a guinea pig, a cat, a dog, a rat, a mouse, a horse, and a whale), or a bird (e.g., a duck or a goose).

As used herein, the terms “treating”, or “treatment” refer to a beneficial or desired result, such as improving one of more, or symptoms of a disease. The terms “treating” or “treatment” also include, but are not limited to, alleviation or amelioration of one or more symptoms of graft rejection described herein. “Treatment” can also mean prolonging survival as compared to expected survival in the absence of treatment.

By “lower” in the context of a disease marker or symptom is meant a clinically-relevant and/or a statistically significant decrease in such level. The decrease can be, for example, at least 10%, at least 20%, at least 30%, at least 40%, or more, down to a level accepted as within the range of normal for an individual without such a disorder. In certain aspects, the decrease is down to a level accepted as within the range of normal for an individual without such disorder which can also be referred to as a normalization of a level. In certain aspects, the reduction is the normalization of the level of a sign or symptom of a disease, a reduction in the difference between the subject level of a sign of the disease and the normal level of the sign for the disease (e.g., to the upper level of normal when the value for the subject must be decreased to reach a normal value, and to the lower level of normal when the value for the subject must be increased to reach a normal level). The term “lowering” may be used interchangeably with “inhibiting,” “reducing,” “silencing,” “downregulating,” “suppressing,” “knocking down,” and other similar terms, and includes any level of inhibition unless otherwise specified.

“Therapeutically effective amount” or an “amount effective” as used herein, is intended to include the amount of a therapeutic agent as described herein that, when administered to a subject having a disease, is sufficient to effect treatment of the disease (e.g., by diminishing, ameliorating, or maintaining the existing disease or one or more symptoms of disease). The “therapeutically effective amount” may vary depending on the nature of the injury and its causes, how the therapeutic agent is administered, the disease and its severity and the history, age, weight, family history, genetic makeup, the types of preceding or concomitant treatments, if any, and other individual characteristics of the subject to be treated. A “therapeutically-effective amount” also includes an amount of a therapeutic agent that produces some desired local or systemic effect at a reasonable benefit/risk ratio applicable to any treatment.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions, such as parenteral or inhaled compositions, in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An “effective amount” or “amount effective” to achieve a desirable therapeutic, pharmacological, medicinal, or physiological effect is any amount that achieves the stated purpose, for example, an amount of an active agent described herein effective to treat cancer or a fibrotic disease. Based on the teachings provided herein, one of ordinary skill can readily ascertain effective amounts of the elements of the described dosage form and produce a safe and effective dosage form and drug product. Because the nature of the active agents described herein varies greatly, and many are repurposed from other uses, a person of ordinary skill would know much about the therapeutic window of each drug, understanding bioavailability, toxic levels, effective targeting levels, etc. and can readily ascertain efficacy of any given dose by monitoring graft rejection parameters, such as transcriptome profiles in PBMC's or graft biopsies, or determining organ function by suitable clinical biomarkers in the patient. PBMC populations may be refined to, CD14, NK, NKT, memory subsets of CD4, CD8, CD3, naïve B-cells, and/or memory B-cell populations. Examples of an effective amount of an active agent compounded in a delivery vehicle includes from 100 μg per ml (picograms per milliliter) to 1 mg/ml (milligrams per milliliter) of solution, including any increment therebetween, such as from 1 ng/ml (nanogram/milliliter) to 1 mg/ml or from 1 ng/ml to 1 μg/ml (microgram/milliliter).

Drug products, or pharmaceutical compositions comprising an active agent (e.g., drug), may be prepared by any method known in the pharmaceutical arts, for example, by bringing into association the active ingredient with the carrier(s) or excipient(s). As used herein, a “pharmaceutically acceptable excipient”, “carrier”, or “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable excipients include one or more of water, saline, phosphate buffered saline, dextrose, glycerol, ethanol, and the like, as well as combinations thereof. In many cases, it may be preferable to include isotonic agents, for example, sugars, polyalcohol's such as mannitol, sorbitol, or sodium chloride in the composition. Pharmaceutically acceptable carriers may further comprise minor amounts of auxiliary substances such as wetting or emulsifying agents, preservatives, or buffers, which enhance the shelf life or effectiveness of the active agent. In certain aspects, the active compound may be prepared with a carrier that will protect the compound against rapid release, such as a controlled release formulation, including implants, transdermal patches, and microencapsulated delivery systems. Biodegradable, biocompatible polymers can be used in delivery systems, such as ethylene vinyl acetate, polyanhydrides, polyglycolic acid, collagen, polyorthoesters, and polylactic acid. Many methods for the preparation of such formulations are broadly-known to those skilled in the art. The preferred form may depend on the intended mode of administration and therapeutic application, which will in turn dictate the types of carriers/excipients. Suitable forms include, but are not limited to, liquid, semi-solid, and solid dosage forms.

Pharmaceutical formulations adapted for oral administration may be presented, for example and without limitation, in capsules, tablets, oral solutions, or the like, and include suitable carriers and coatings as are broadly-known in the pharmaceutical arts.

Pharmaceutical formulations adapted for parenteral administration may be presented, for example and without limitation, in syringes, vials, bottles, IV/infusion bags, or the like, as are broadly-known to those of ordinary skill. Excipients include, for example and without limitation, water, saline, PBS, lactated Ringers, or any other injectable carriers. Suitable emulsifiers, lipids, surfactants, or the like may be utilized to maintain an active agent in solution.

Pharmaceutical formulations adapted for transdermal administration may be presented, for example and without limitation, as discrete patches intended to remain in intimate contact with the epidermis of the recipient for a prolonged period of time or electrodes for iontophoretic delivery.

Pharmaceutical formulations adapted for topical administration may be formulated, for example and without limitation, as ointments, creams, suspensions, lotions, powders, solutions, pastes, gels, sprays, aerosols, or oils.

Therapeutic compositions typically must be sterile and stable under the conditions of manufacture and storage. For example, sterile injectable solutions can be prepared by incorporating the active agent in the required amount in an appropriate solvent with suitable carrier(s), followed by filter-sterilization. An appropriate fluidity of a solution can be maintained, for example, by the use of a rheology modifier. Prolonged absorption of injectable compositions can be brought about by including in the composition an agent that delays absorption, for example, monostearate salts and gelatin.

The phrase “pharmaceutically-acceptable carrier” as used herein means a pharmaceutically-acceptable material, composition or vehicle, such as a liquid or solid filler, diluent, excipient, manufacturing aid (e.g., lubricant, talc magnesium, calcium, zinc stearate, or steric acid), or solvent encapsulating material, involved in carrying or transporting the subject compound from one organ, or portion of the body, to another organ, or portion of the body.

The therapeutic agents described herein can be administered by any effective route. Examples of delivery routes include, without limitation: topical, for example, epicutaneous, inhalational, enema, ocular, otic, and intranasal delivery; enteral, for example, orally, by gastric feeding tube, and rectally; and parenteral, such as, intravenous, intraarterial, intrathecally, intramuscular, intracardiac, subcutaneous, intraosseous, intradermal, intrathecal, intraperitoneal, transdermal, iontophoretic, transmucosal, epidural, and intravitreal, with intrathecal and oral approaches being preferred in many instances. Suitable dosage forms may include single-dose, or multiple-dose vials or other containers, such as medical syringes, containing a composition comprising the therapeutic agent useful for treatment of graft rejection as described herein.

Dosage regimens may be adjusted to provide the optimum desired response (e.g., a therapeutic or prophylactic response). For example, a single bolus may be administered, several divided doses may be administered over time, or the therapeutic agent may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate therapeutic agents in dosage unit form for ease of administration and uniformity of dosage. The specification for the dosage unit forms may be dictated by and directly dependent on (a) the unique characteristics of the therapeutic agent and the particular therapeutic or prophylactic effect to be achieved, and (b) the limitations inherent in the art of compounding such a therapeutic agent for the treatment of sensitivity in individuals.

By “target-specific” or reference to the ability of one compound to bind another target compound specifically, it is meant that the compound binds to the target compound to the exclusion of others in a given reaction system, e.g., in vitro, or in vivo, to acceptable tolerances, permitting a sufficiently specific diagnostic or therapeutic effect according to the standards of a person of skill in the art, a medical community, and/or a regulatory authority, such as the U.S. Food and Drug Agency (FDA), in aspects, in the context of administering a reagent as described herein to LF tissue in a patient.

By “expression” or “gene expression,” it is meant the overall flow of information from a gene. A “gene” is a sequence of DNA or RNA which codes for a molecule, such as a protein or a functional RNA, such as an ncRNA that has a function. A “gene” is a functional genetic unit for producing its gene product, such as RNA or a protein in a cell, or other expression system encoded on a nucleic acid and generally comprising: a transcriptional control sequence, such as a promoter and other cis-acting elements, such as transcriptional response elements (TREs) and/or enhancers; an expressed sequence that typically encodes a protein (referred to as an open-reading frame or ORF) or functional/structural RNA; and a polyadenylation sequence). A gene produces a gene product (typically a protein, optionally post-translationally modified, or a functional/structural RNA) when transcribed. By “expression of genes under transcriptional control of,” or alternately “subject to control by” a designated sequence such as a promotor, it is meant gene expression from a gene containing the designated sequence operably linked (functionally attached, typically in cis) to the gene. A gene that is “under transcriptional control” of a promotor or transcription control element, is a gene that is transcribed at detectably different levels in the presence of a transcription factor, e.g., in specific cells, as further described below, and in the context of the present disclosure, produces a difference in transcription levels when expressed in a specific cell type. A “gene for expression of” a stated gene product is a gene capable of expressing that stated gene product when placed in a suitable environment, that is, for example, when transformed, transfected, transduced, etc. into a cell, and subjected to suitable conditions for expression. In the case of a constitutive promoter “suitable conditions” means that the gene typically need only be introduced into a host cell. In the case of an inducible promoter, “suitable conditions” means when factors that regulate transcription, such as DNA-binding proteins, are present or absent, for example, an amount of the respective inducer is available to the expression system (e.g., cell), or factors causing suppression of a gene are unavailable or displaced-effective to cause expression of the gene.

In further detail, transcription is the process by which the DNA gene sequence is transcribed into RNA. The steps include transcript initiation, transcript elongation, and transcript termination. The molecular machinery of transcription includes but is not limited to: RNA polymerase, general transcription factors, enhancers, and promoter DNA, and RNA transcript. Transcription factors (TFs) are proteins that control the rate of transcription of genetic information from DNA to RNA, by binding to a specific DNA sequence (i.e., the promoter region). The function of TFs is to regulate genes in order to make sure that they are expressed in the right cell at the right time and in the right amount throughout the life of the cell and the organism. The promoter region of a gene is a region of DNA that initiates transcription of that particular gene. Promoters are located near the transcription start sites of genes, on the same strand, and often, but not exclusively, are upstream (towards the 5′ region of the sense strand) on the DNA. Promoters can be about 100-1000 base pairs long. Additional sequences and non-coding elements can affect transcription rates. If the cell has a nucleus (eukaryotes), the RNA is further processed. This includes polyadenylation, capping, and splicing. Polyadenylation is the addition of a poly (A) tail to a messenger RNA. The poly (A) tail consists of multiple adenosine monophosphates; in other words, it is a stretch of RNA that has only adenine bases. In eukaryotes, polyadenylation is part of the process that produces mature messenger RNA (mRNA) for translation. Capping refers to the process wherein the 5′ end of the pre-mRNA has a specially altered nucleotide. In eukaryotes, the 5′ cap (cap-0), found on the 5′ end of an mRNA molecule, consists of a guanine nucleotide connected to mRNA via an unusual 5′ to 5′ triphosphate linkage. During RNA splicing, pre-mRNA is edited. Specifically, during this process introns are removed, and exons are joined together. The resultant product is known as mature mRNA. The RNA may remain in the nucleus or exit to the cytoplasm through the nuclear pore complex.

Gene expression involves various steps, including transcription, post-transcriptional RNA modification, translation, and post-translational modification of a protein. Expression of a gene may also include reduction of the total amount of the protein product, such as by cleavage, sequestration, binding, or other means of decreasing the function or amount of a protein product.

Complementary refers to the ability of polynucleotides (nucleic acids) to hybridize to one another, forming inter-strand base pairs. Base pairs are formed by hydrogen bonding between nucleotide units in antiparallel polynucleotide strands. Complementary polynucleotide strands can base pair (hybridize) in the Watson-Crick manner (e.g., A to T, A to U, C to G), or in any other manner that allows for the formation of duplexes. When using RNA as opposed to DNA, uracil rather than thymine is the base that is complementary to adenosine. Two sequences comprising complementary sequences can hybridize if they form duplexes under specified conditions, such as in water, saline (e.g., normal saline, or 0.9% w/v saline or phosphate-buffered saline), or under other stringency conditions, such as, for example and without limitation, 0.1×SSC (saline sodium citrate) to 10×SSC, where 1×SSC is 0.15M NaCl and 0.015M sodium citrate in water. Hybridization of complementary sequences is dictated, (e.g., by salt concentration and temperature), with the melting temperature (Tm) lowering with increased mismatches and increased stringency. Perfectly matched sequences are said to be fully complementary or have 100% sequence identity (gaps are not counted, and the measurement is in relation to the shorter of the two sequences). In one aspect, a sequence that “specifically hybridizes” to another sequence, does so in a hybridization solution containing 0.5M sodium phosphate buffer, pH 7.2, containing 7% SDS, 1 mM EDTA, and 100 mg/ml of salmon sperm DNA at 65° C. for 16 hours and washing twice at 65° C. for twenty minutes in a washing solution containing 0.5×SSC and 0.1% SDS, or does so under conditions more stringent than 2×SSC at 65° C., for example, in 0.2×SSC at 55° C. A sequence that specifically hybridizes to another typically has at least 80%, 85%, 90%, 95%, or 99% sequence identity with the other sequence. For purposes of comparing sequence identity of an RNA sequence to that cDNA sequence, Ts and Us are interchangeable.

The term “homology” can refer to a percent (%) identity of a nucleic acid or polypeptide to a reference nucleic acid or polypeptide. As a practical matter, whether any particular RNA can be at least 50%, 60%, 70%, 80%, 85%, 90%, 92%, 95%, 96%, 97%, 98%, or 99% identical to any reference nucleic acid or polypeptide sequence of any nucleic acid or polypeptide described herein (which may correspond with a particular nucleic acid sequence described herein), such particular nucleic acid or polypeptide sequence can be determined conventionally using known computer programs such the Bestfit program (Wisconsin Sequence Analysis Package). When using Bestfit or any other sequence alignment program to determine whether a particular sequence is, for instance, 95% identical to a reference sequence according to the present invention, the parameters can be set such that the percentage of identity is calculated over the full length of the reference nucleic acid or polypeptide sequence and that gaps in homology of up to 5% of the total number of nucleic acid or polypeptide bases in the reference sequence are allowed.

Production of useful nucleic acid constructs, such as the genetic constructs and recombinant viral genomes described herein, is routine, in that molecular cloning and gene assembly methods are routine. Further, a number of companies can custom-synthesize and verify multi-kilobase genes, making the production of genes or genomes as described herein, such as recombinant viral genomes, routine (See, e.g., Gene Synthesis Handbook, 2d Edition, 2014, GenScript USA, Inc.).

Provided herein are antigen binding molecules, e.g., antibody compounds comprising an antibody domain targeting uPAR proteins and epitopes, and methods of use of those compositions. The term “antigen binding molecule”, for ease of reference and unless otherwise specified, refers to an immunoglobulin, derivatives thereof which maintain specific binding ability, and proteins having an antigen-binding domain which is homologous or largely homologous to an immunoglobulin binding domain, and complexes thereof, which are typically covalently linked, as in immunoglobulin (see, e.g., Chailyan A, Marcatili P, Tramontano A. The association of heavy and light chain variable domains in antibodies: implications for antigen specificity. FEBS J. 2011 August; 278 (16): 2858-66, and U.S. Pat. No. 11,578,428 B2, and US Patent Publication No. 2024/0158529, showing typical antibody structures, including humanized antibodies). As such, the antigen binding molecule operates as a ligand for its cognate antigen, which can be virtually any polypeptide or protein. Natural antibodies typically comprise two heavy chains and two light chains and are bi-valent. The interaction between the variable regions of heavy and light chain forms a binding site (e.g., a paratope, defined by a set of CDRs) capable of specifically binding an antigen. The term “V_{H}” refers to a heavy chain variable region of an antibody. The term “V_{L}” refers to a light chain variable region of an antibody. Antibodies may be derived from natural sources, or partly or wholly synthetically produced, and may be “humanized” to reduce immunogenicity, as is known in the related arts. An antibody may be monoclonal or polyclonal. An antibody may be a member of any immunoglobulin class, including, for example and without limitation, any of the human classes: IgG, IgM, IgA, IgD, and IgE.

An antigen binding molecule or complexes thereof may be, for example and without limitation, a monoclonal antibody, including fragments, derivatives, or analogs thereof, or complexes thereof, including without limitation: Fab, Fab′, Fv fragments, single chain Fv (scFv) fragments, dsFv, Fab₁ fragments, F(ab′)₂ fragments, single domain antibodies, camelized (camelid) antibodies and antibody fragments, humanized antibodies and antibody fragments, and multivalent versions of the foregoing; multivalent binding reagents including without limitation: monospecific or bispecific antibodies, such as disulfide stabilized Fv fragments, scFv tandems ((ScFv)₂ fragments), diabodies, triabodies, tetrabodies, which typically are covalently linked or otherwise stabilized (e.g., leucine zipper or helix stabilized) scFv fragments, bi-specific T-cell engager (BiTE, e.g., a DbTE), di-scFv (dimeric single-chain variable fragment), single-domain antibody (sdAb), or antibody binding domain fragments. Antibody fragments also include miniaturized antibodies or other engineered binding reagents that exploit the modular nature of antibody structure, comprising, often as a single chain, one or more antigen-binding or epitope-binding sequences (e.g., paratope) and, at a minimum, any other amino acid sequences needed to ensure appropriate specificity, delivery, and stability of the composition.

The binding reagents described herein, comprise, at their core single-chain V_{H} paratopes, that are defined by three CDR's (typically loops), CDR1, CDR2, and CDR3, which for V_{H} peptides may be termed HCDR1, HCDR2, and HCDR3, respectively, and which are flanked by, and separated by framework (e.g., joining or scaffold) amino acid sequences that space apart and support the CDRs, and which may differ from antibody-to-antibody, and which may be “humanized” to minimize antigenicity when administered to a human patient. In nature, HCDR3 is typically the most variable of the CDRs, contributing significantly to antibody specificity. Various methods may be used to identify the precise limits of each CDR, but the sequences provided herein can be evaluated by any suitable method to determine the CDRs.

Exemplary CDR sequences of V_{H} 3 include:

VH3-HCDR1:
(SEQ ID NO: 13)
RYWMS;
VH3-HCDR2:
(SEQ ID NO: 14)
EINHSGSTNYNPSLKS;
and
VH3-HCDR3:
(SEQ ID NO: 15)
SLVPALSYYYYYGMDV.

Exemplary CDR sequences of V_{H} 115 include:

VH115-HCDR1:
(SEQ ID NO: 16)
DYAIG;
VH115-HCDR2:
(SEQ ID NO: 17)
WINTNSGSPKYAQGFTG;
and
VH115-HCDR3:
(SEQ ID NO: 18)
DVVVP.

CDR sequences may be determined using searches described in abYsis (abysis.com). Both VH3 and VH115 are derived from germline V_{H} clone IGHV3-23 (IGHV3-23 immunoglobulin heavy variable 3-23 [Homo sapiens (human)] Gene ID: 28442, also, UniProt P01764). Other methods of CDR identification are known in the art (see, e.g., Kunik V, Ashkenazi S, Ofran Y. Paratome: an online tool for systematic identification of antigen-binding regions in antibodies based on sequence or structure. Nucleic Acids Res. 2012 July; 40 (Web Server issue): W521-4; Adolf-Bryfogle J, Xu Q, North B, Lehmann A, Dunbrack R L Jr. PylgClassify: a database of antibody CDR structural classifications. Nucleic Acids Res. 2015 January; 43 (Database issue): D432-8), and assorted online tools and applications as are broadly-available. As such, an amino acid sequence of a V_{H} or V_{L} may be provided or determined, comprising CDRs, and one of ordinary skill can determine the precise metes and bounds of CDRs within that antibody sequence without undue experimentation. Framework sequences may be optimized (see, e.g., Gopal R, Fitzpatrick E, Pentakota N, Jayaraman A, Tharakaraman K, Capila I. Optimizing Antibody Affinity and Developability Using a Framework-CDR Shuffling Approach-Application to an Anti-SARS-CoV-2 Antibody. Viruses. 2022 Nov. 30; 14 (12): 2694) and/or humanized based on knowledge of amino acid sequences of the CDRs, e.g., HCDR1, HCDR2, and HCDR3 of V_{H} 3 and V_{H}115 provided herein.

Antibodies may be produced by any effective method, such as by hybridoma or it may be recombinantly or synthetically produced. In the context of the present disclosure and for ease of reference, “antibodies” or “antibody” may refer to both natural antibodies as well as protein antibody analogs, antibody fragments, and derivatives, any of which comprising V_{L} and/or V_{H} sequences and/or CDRs (e.g., all three CDRs of any VH or V_{L} region, defining a paratope as described herein) according to any example, aspect, or embodiment described herein. The antibody or antibodies may be synthetic, in that they do not comprise a naturally-occurring sequence, such as the VH 3 and VH115 antigen binding molecules, including engineered versions and derivatives thereof, such as scFv versions thereof, humanized versions thereof, BiTEs, and/or sequence derivatives thereof, including without limitation, proteins comprising the CDRs (e.g., one or more, or all three CDRs) of VH 3 and VH115.

Nanobodies, which may be referred to a V_{HH} antibodies or single-domain antibodies, may be constructed using CDR sequences, such as the VH 3 and VH 115 CDRs as provided herein. A V_{H}, alone, may be capable of defining an antigen-binding site (e.g., paratope) with sufficient strength to be pharmacologically-useful, as with the VH 3 and VH 115 CDRs described herein, and can be referred to as a nanobody (see, e.g., Wesolowski J, Alzogaray V, Reyelt J, Unger M, Juarez K, Urrutia M, Cauerhff A, Danquah W, Rissiek B, Scheuplein F, Schwarz N, Adriouch S, Boyer O, Seman M, Licea A, Serreze D V, Goldbaum F A, Haag F, Koch-Nolte F. Single domain antibodies: promising experimental and therapeutic tools in infection and immunity. Med Microbiol Immunol. 2009 August; 198 (3): 157-74, providing structure and sequences of various nanobodies and Bever C S, Dong J X, Vasylieva N, Barnych B, Cui Y, Xu Z L, Hammock B D, Gee S J. V_{HH} antibodies: emerging reagents for the analysis of environmental chemicals. Anal Bioanal Chem. 2016 September; 408 (22): 5985-6002). Single-domain antigen binding molecules originally were camelid antibodies, which naturally comprise only heavy chains (see, e.g., Mitchell L S, Colwell L J. Comparative analysis of nanobody sequence and structure data. Proteins. 2018 July; 86 (7): 697-706). More recently other single-chain antigen-binding molecules have been developed. Construction and humanization of single-domain antigen binding molecules is broadly-known (see, e.g., Valdes-Tresanco M S, Molina-Zapata A, Pose A G, Moreno E. Structural Insights into the Design of Synthetic Nanobody Libraries. Molecules. 2022 Mar. 28; 27 (7): 2198; Ahmad Z A, Yeap S K, Ali A M, Ho W Y, Alitheen N B, Hamid M. scFv antibody: principles and clinical application. Clin Dev Immunol. 2012; 2012:980250. doi: 10.1155/2012/980250; Wu Y, Jiang S, Ying T. Single-Domain Antibodies As Therapeutics against Human Viral Diseases. Front Immunol. 2017 Dec. 13; 8:1802; Hoey R J, Eom H, Horn J R. Structure and development of single domain antibodies as modules for therapeutics and diagnostics. Exp Biol Med (Maywood). 2019 December; 244 (17): 1568-1576; Rossotti M A, Bélanger K, Henry K A, Tanha J. Immunogenicity and humanization of single-domain antibodies. FEBS J. 2022 July; 289 (14): 4304-4327; and Khodabakhsh F, Behdani M, Rami A, Kazemi-Lomedasht F. Single-Domain Antibodies or Nanobodies: A Class of Next-Generation Antibodies. Int Rev Immunol. 2018; 37 (6): 316-322). Multimerization methods are broadly-known, too (see, e.g., Miller A, Carr S, Rabbitts T, Ali H. Multimeric antibodies with increased valency surpassing functional affinity and potency thresholds using novel formats. MAbs. 2020 January-Dec; 12 (1): 1752529), for example to produce bi-specific antibody binding molecules, such as bi-specific T-cell engagers (e.g., BiTEs), discussed in further detail, below. Nanobody construction has been commercialized, e.g. in Crescendo Biologics' Humabody platform (see, Teng Y, et al., Diverse human VH antibody fragments with bio-therapeutic properties from the Crescendo Mouse. N Biotechnol. 2020 Mar. 25; 55:65-76, U.S. Pat. No. 11,547,099 B2, and WO 2016/062988 for exemplary constructs, transgenic mice, and methods for producing V_{H} nanobodies).

CAR cells, including CAR T and CAR NK cells, are synthetic cells that bear recombinant surface receptors engineered to bind surface proteins on target cells, such as cancer cells. CAR T cells can use a patient's T cells as a starting material. CAR NK may include engineered cells derived from functional NK cells. Commonly used sources of functional NK cells for CAR-NK production include the NK-92 cell line, adult peripheral blood, umbilical cord blood, and induced pluripotent stem cells (see, e.g., Moscarelli J, Zahavi D, Maynard R, Weiner L M. The Next Generation of Cellular Immunotherapy: Chimeric Antigen Receptor-Natural Killer Cells. Transplant Cell Ther. 2022 October; 28 (10): 650-656). A CAR construct comprises three components: an ectodomain for antigen recognition e.g., a VH 3 CDR- or VH 115 CDR-containing amino acid sequence, a transmembrane linker, and an intracellular signaling domain, such as a CD32 and/or other co-stimulatory domains, such as CD28, 4-1BB, 2B4, DAP-10, or DAP-12, and CAR cells may also comprise additional recombinant genes for expression of payloads, such as IL-7, IL-12, IL-15, IL-18, and/or IL-23 (see, e.g., Moscarelli et al.; Sterner R C, Sterner R M. CAR-T cell therapy: current limitations and potential strategies. Blood Cancer J. 2021 Apr. 6; 11 (4): 69; Wang G, Zhou X, Fuca G, Dukhovlinova E, Shou P, Li H, Johnston C, Mcguinness B, Dotti G, Du H. Fully human antibody VH domains to generate mono and bispecific CAR to target solid tumors. J Immunother Cancer. 2021 April; 9 (4): e002173; Zhenyu Dai, Wei Mu, Ya Zhao, Jiali Cheng, Haolong Lin, Kedong Ouyang, Xiangyin Jia, Jianwei Liu, Qiaoe Wei, Meng Wang, Chaohong Liu, Taochao Tan2 and Jianfeng Zhou. T cells expressing CD5/CD7 bispecific chimeric antigen receptors with fully human heavy-chain-only domains mitigate tumor antigen escape. Signal Transduction and Targeted Therapy. 2022, 7:85).

BiTE (bi-specific T cell engagers, e.g., domain-based T cell engagers, or DbTEs) comprise an antigen binding molecule that binds a target antigen, e.g., uPAR, and T-cells, and recruit T-cells to target-antigen-expressing cells (see, e.g., Kegyes D, Constantinescu C, Vrancken L, Rasche L, Gregoire C, Tigu B, Gulei D, Dima D, Tanase A, Einsele H, Ciurea S, Tomuleasa C, Caers J. Patient selection for CAR T or BiTE therapy in multiple myeloma: Which treatment for each patient? J Hematol Oncol. 2022 Jun. 7; 15 (1): 78; Tian Z, Liu M, Zhang Y, Wang X. Bispecific T cell engagers: an emerging therapy for management of hematologic malignancies. J Hematol Oncol. 2021 May 3; 14 (1): 75; Zhou S, Liu M, Ren F, Meng X, Yu J. The landscape of bispecific T cell engager in cancer treatment. Biomark Res. 2021 May 26; 9 (1): 38; and Wang et al. Fully human antibody VH domains to generate mono and bispecific CAR to target solid tumors. J Immunother Cancer. 2021 April; 9 (4): e002173).

An antigen-drug conjugate (ADC) is provided herein. A uPAR-binding antigen binding molecule according to any aspect, embodiment, or example provided described herein may be linked to a payload (e.g., a cargo or warhead) that causes a desired physiological effect, such as killing a cell expressing uPAR on its surface. As shown herein, the anti-uPAR V_{H} molecules, e.g., VH3:

(EVQLVESGGGLVQPGGSLRLSCAASGFTFSRYWMSWVRQAPGKALEWI
GEINHSGSTNYNPSLKSLVTISRDNSKNTLYLQMNSLRAEDTATYYCAR
SLVPALSYYYYYGMDVWGQGTTVTVSS (SEQ ID NO: 1)),
or

### V_{H}115:

(EVQLVESGGGLVQPGGSLRLSCKGSGFTFGDYAIGWVRQAPGQRLEWI
GWINTNSGSPKYAQGFTGRFTISRDNSKNTLYLQMNSLRAEDTAVYYCA
TDVVVPWGQGSQVTVSS (SEQ ID NO: 3)),

or a sequence having at least 70%, at least 75%, at least 80%, at least 90%, at least 95% or 100% sequence identity with VH3 (SEQ ID NO: 1) or VH115 (SEQ ID NO: 3) can be effectively covalently-linked to other moieties, with Fc and DbTE sequences being exemplary proof of concept yet retain significant antigen-binding capacity. ADCs comprise an antigen binding moiety (linked antigen-binding molecule), a linker that may be cleavable or non-cleavable, and the payload moiety. A “moiety” is a chemical group or entity, often functional, that forms part of a larger molecule. Linkers may be used to join a payload moiety to the antigen binding molecule, and choice of linkers can depend on how the APC is handled by the cell, and how the payload becomes effective on processing by a cell, or by release due to chemical lability of the linker. Non-cleavable linkers include, without limitation, alkyl moieties, and thioether moieties (e.g., Succinimidyl 4-(N-maleimidomethyl) cyclohexane-1-carboxylate (SMCC)). Cleavable or labile linkers may include, for example and without limitation, acid-labile linkers (hydrolysable in lysosomes or endosomes), Lysosomal protease-sensitive linkers (e.g., peptide-based linkers), 3-glucuronide linkers, and glutathione-sensitive disulfide linkers, with examples including, without limitation: ester-, hydrazone-, Valine-citrulline (v-c)-, Valine-alanine (v-a)-, and phenylalanine-lysine (p-I)-containing linkers. (see, e.g., Khongorzul P, Ling C J, Khan F U, Ihsan A U, Zhang J. Antibody-Drug Conjugates: A Comprehensive Review. Mol Cancer Res. 2020 January; 18 (1): 3-19, describing exemplary linking methods and suitable cytotoxic payloads or warheads). Examples of payloads include, without limitation: microtubule-disrupting agents, such as auristatin, maytansinoids, eribulin (e.g., eribulin mesylate), tubulysins, cryptophycins, and EG5 inhibitors; DNA-damaging agents, such as, without limitation calicheamicin, duocarmycins, doxorubicin, enediyne, topoisomerase I inhibitors, and Pyrrolo[2,1-c][1,4] benzodiazepines; RNA-targeting payloads, such as thailanstatins and amatoxins; immune payloads, such as Toll-like receptor agonists, STING agonists, glucocorticoid receptor modulators; and other payloads, such as Bcl-xL inhibitors, NAMPT inhibitors, and proteasome inhibitors such as carmaphycins. Design and optimization considerations for production of ADCs are provided in Khongorzul P, et al. (Khongorzul P, Ling C J, Khan F U, Ihsan A U, Zhang J. Antibody-Drug Conjugates: A Comprehensive Review. Mol Cancer Res. 2020 January; 18 (1): 3-19, describing exemplary linking methods and suitable cytotoxic payloads or warheads, and see, e.g., Gogia P, Ashraf H, Bhasin S, Xu Y. Antibody-Drug Conjugates: A Review of Approved Drugs and Their Clinical Level of Evidence. Cancers (Basel). 2023 Jul. 30; 15 (15): 3886; Baah S, Laws M, Rahman K M. Antibody-Drug Conjugates-A Tutorial Review. Molecules. 2021 May 15; 26 (10): 2943; and Wang Z, Li H, Gou L, Li W, Wang Y. Antibody-drug conjugates: Recent advances in payloads. Acta Pharm Sin B. 2023 October; 13 (10): 4025-4059). ADCs with multiple payloads, PROTAC-guided ADCs, ADCs with peptide-drug-conjugates, and ADCs with photo-reactive payloads also may be produced. As such a person of ordinary skill can produce, based on the teachings herein and without undue experimentation, an ADC comprising an antigen binding molecule as described herein linked to a cytotoxic payload via a chemical linker.

Unless indicated otherwise, nucleic acid sequences are provided in 5′ to 3′ orientation, and amino acid sequences are provided in an N to C-terminal orientation. Reference to a CDR herein may refer to a Kabat CDR numbering scheme or any other applicable CDR numbering scheme, including but not limited to Chothia, Martin (enhanced Chothia), Gelfand, IMGT, Honneger, or any other numbering scheme (see, e.g., Dondelinger M, et al. Understanding the Significance and Implications of Antibody Numbering and Antigen-Binding Surface/Residue Definition. Front Immunol. 2018 Oct. 16; 9:2278).

Reference to an antigen binding molecule, e.g., a VH 3 and VH115 antigen binding molecule, refers to an antigen binding molecule comprising one, two, or all three CDRs of VH 3 and VH115, such as all three HCDR1, HCDR2, HCDR3 of VH 3 or of VH115 are included, and are spaced apart and flanked by suitable framework amino acid sequences such that the antigen binding molecule retains affinity and avidity for the uPAR antigen, e.g., uPAR epitopes.

While specific types of antigen binding molecules, such as single-chain V_{H} antigen binding molecules, V_{H}Fc molecules, and domain-specific T-cell engagers are described specifically herein, any antigen binding molecule comprising the VH3 or VH115 CDR sequences, for binding uPAR, e.g., in an affinity assay, such as an ELISA assay, bilayer interferometry (e.g., BLItz, see, e.g., Müller-Esparza H, Osorio-Valeriano M, Steube N, Thanbichler M, Randau L. Bio-Layer Interferometry Analysis of the Target Binding Activity of CRISPR-Cas Effector Complexes. Front Mol Biosci. 2020 May 27; 7:98), surface plasmon responance (SPR), oblique-incidence reflectivity difference (OI-RD) binding affinity, or cell binding assay, are contemplated. Antigen binding molecules with high binding affinities may bind to their corresponding antigen with a K_{D} of 100 nM or less, 75 nM or less, 50 nM or less, or 25 nM or less.

Antigen binding molecules described herein are capable of binding uPAR and killing uPAR-expressing cells. While suitable antigen binding molecules are presented herein, further maturation (e.g., mutation and screening for examples by methods described herein) are expected to optimize avidity of any form of the antigen binding molecules and antigen-binding sequences disclosed herein. Two fully human V_{H} domain antibodies that target uPAR we selected and characterized. Both antibodies showed high affinity for uPAR. Converting the antibodies to VH-Fc fusion protein enhanced the avidity of VH 3 by 2-fold, but decreased avidity 40-fold in VH 115, which may be due to aggregation, and which may be resolved by further antibody maturation of VH 115. DbTEs based on VH 3 and VH 15 showed specific cell killing of cells with observable expression levels of uPAR, demonstrating their potential for cancer, antifibrotic, and/or senolytic therapies.

Methods of killing uPAR-expressing cells are provided herein, with potential therapies in, for example and without limitation, cancers, fibrotic disease, senescence-related disease, or any disease caused or characterized by cells exhibiting higher than normal uPAR expression. Effective amounts of uPAR antigen binding molecules, or cells such as T-cells or NK cells expressing chimeric antigen receptors including the uPAR antigen binding molecules, may be administered to a patient, or to cells in an in vivo or ex vivo cell culture to kill the uPAR-expressing cells. The uPAR expressing cells may be associated with a disease, such as cancer cells such as tumor or blood cancer cells, or senescent cells. Methods of treatment of cancer are therefore provided, including treatment of breast cancer, colorectal cancer, melanoma, brain cancer, lung cancer, ovarian cancer, prostate cancer, bladder cancer, liver cancer, gastric cancer, pancreatic cancer, gliomas, or hematologic malignancies, such as acute myeloid leukemia or myeloma Methods of treatment of fibrotic diseases are therefore provided, including treatment of systemic sclerosis (SSc), idiopathic pulmonary fibrosis, urinary tract fibrosis (e.g., renal fibrosis, ureteral fibrosis, urethral fibrosis, bladder fibrosis, or Peyronie's disease), liver cirrhosis, or COPD. In treatment of diseases (e.g., fibrotic disease) or cancers, effective amounts of uPAR antigen binding molecules, or cells such as T-cells or NK cells expressing chimeric antigen receptors including the uPAR antigen binding molecules, may be administered to a patient, or to cells in an in vivo or ex vivo cell culture to kill the uPAR-expressing cells or to otherwise treat the disease (e.g., fibrotic disease) or cancer.

### Example—Human Antibody V_{H} Domains Targeting uPAR as Candidate Therapeutics for Cancers and Fibrotic Disease

The high expression of uPAR has been linked to tumor progression, invasion, and metastasis in several types of cancer. Such overexpression of uPAR makes it a potential target for immunotherapies across common cancers such as breast, colorectal, lung, ovarian cancer, and melanoma. In our study, two high-affinity and specific human V_{H} domain antibody candidates, designed as clones 3 and 115, were isolated from a phage-displayed human V_{H} antibody library. Domain-based bispecific T-cell engagers (DbTE) based on these two antibodies exhibited potent killing of uPAR-positive cancer cells. Thus, these two anti-uPAR domain antibodies are promising candidates for treating uPAR positive cancers.

Further to the above, urokinase-type plasminogen activator receptor (uPAR), also named CD87, is a single-chain membrane glycoprotein receptor containing three homologous domains (D1, D2, and D3) anchored to the cell membrane by a GPI linkage. In normal physiological conditions, uPAR expression is fairly low. This expression, however, can be highly elevated in many types of cancer including breast cancer, colorectal cancer, melanoma, brain cancer, lung cancer, ovarian cancer, prostate cancer, liver cancer, gastric cancer, and pancreatic cancer. uPAR also plays an important role in tumor proliferation, metastasis, angiogenesis, and prognosis. Many studies have revealed that high expression of uPAR is related to poor prognosis and that expression level can serve as a marker of tumor malignancy. Even though several systems are involved, the uPA-uPAR signaling pathway plays a central role from tumor proliferation to metastasis.

The uPA-uPAR-α5β1 integrin complex can bind to G-protein-coupled receptors (GPCRs) or interact with EGFR or PGGFRβ to activate focal adhesion kinase (FAK)-MAPK-ERK pathway and PI3K/AKT pathway, which promotes tumor cell proliferation and survival. uPAR's expression on the non-malignant cells that infiltrate cancers and on malignant tumor cells adds to its importance in tumor progression and poor prognosis. When uPA is activated after binding to uPAR, plasminogen is cleaved into active plasmin, which further activates MMPs to degrade ECM and regulate cell migration. Moreover, uPAR interacts with VEGFR2 and promotes VEGFR2 internalization, thus, enhancing the VEGF-induced angiogenesis. Taken together, uPAR's expression in cancer and importance in tumor make the receptor an attractive therapeutic target for cancer treatment, in addition to prognosis and diagnosis.

The development of monoclonal antibody-based immunotherapy has opened new avenues to specifically target cancer cells expressing certain receptors. Such therapies have become an increasingly attractive option for cancer treatment due to its high efficacy and lower side effects compared to other options such as surgery, radiation, and chemotherapy. In addition to the binding to targeted antigens, antibodies can also mobilize anti-tumor immunity through effector functions. Different antibody structures have been developed in recent decades, including fragment antigen-binding region (Fab), single-chain Fv (scFv) fragments, and domain antibodies (V_{H} and V_{L}), for tailored applications. Among these formats, antibody heavy chain variable (V_{H}) domains have shown increasing promise in antibody-based cancer immunotherapy due to their small size (ranging from 11 kDa to 15 kDa), high affinity, high yields, and low immunogenicity. Studies have shown that these lower molecular weight proteins can deeply penetrate tissues, and enabling immunotherapies to target new epitopes that are not accessible to large antibody constructs. Thus, the use of variable domain antibodies may prove useful in the development of cancer immunotherapies, especially for solid tumors.

Two potent human V_{H} domain antibodies that target human uPAR are described below. These binders were characterized for their affinity and specificity. The domain-based bispecific T cell engager (DbTE) based on these two binders showed potent killing effects of uPAR-expressing cancer cells. To our knowledge, this is the first report of uPAR-specific human V_{H} domain antibodies as candidates for cancer immunotherapy.

### Materials and Methods

Panning of High-Affinity V_{H} Domains against uPAR from Large V_{H} Phage Library: Human uPAR-Fc (Catalog #10378-UK-100) and uPAR-His (Catalog #807UK100CF) recombinant proteins were purchased from the R&D system. To pan antibody candidates against uPAR, a large phage-displayed human V_{H} library was used against human IgG1 Fc fused recombinant uPAR. After the first round of panning against 5 μg uPAR-Fc, two additional rounds of panning were performed using progressively 1-fold smaller quantities of uPAR-Fc to increase selective pressure. 192 individual clones obtained from the final round of panning were screened for binding to uPAR-His protein by ELISA.

Expression and purification of V_{H}, V_{H}-Fc, and DbTE: To convert V_{H} antibody candidates to V_{H}-Fc format, the V_{H} domain was amplified and cloned into the pcDNA-IgG1 Fc vector. For the construction of DbTE, humanized OKT3 scFv (VH-(G₄S) ₆-VL (SEQ ID NO: 19)) was inserted at the C terminal of V_{H} followed by the IgG1 Fc with LALAPG (SEQ ID NO: 20) mutation. Both the V_{H}-Fc and DbTE were transiently transfected and expressed by the Expi293 expression system, then purified by protein A resin (Thermo Fisher). The V_{H} binder was expressed in E. coli TopF expression system and purified on Ni-NTA columns (GE Healthcare).

ELISA: The binding and specificity of V_{H}, V_{H}-Fc, and DbTE to uPAR or CD3 were analyzed by ELISA. uPAR-His protein or CD3 protein was coated at 50 ng/well at 4° C. overnight, then blocked with 5% milk for 1 hours at 37° C. After washing 3 times by 0.05% PBST, 3-fold serially diluted V_{H} and V_{H}-Fc binders were incubated on the plate for 1 hour at 37° C. The binding of V_{H} candidates was detected by anti-FLAG M2-peroxidase (HRP) antibody (Sigma-Aldrich) while V_{H}-Fc or DbTE binding was detected by HRP conjugated goat anti-human IgG1 Fc (Sigma-Aldrich) at 1:1000 dilution. The plates were washed 3 times by 0.05% PBST between each reagent's incubation. Binding activity was detected using 3,3′,5,5′-tetramethylbenzidine (Sigma-Aldrich) and was stopped by TMB stop buffer (ScyTek Laboratories). Absorbance was read at 450 nm.

BLItz: DPBS was used to establish a baseline for 30 s. Streptavidin biosensors (ForteBio) were coated with 16.7 μg/mL recombinant uPAR-Biotin for 2 min. For competition assay, 500 nM of V_{H} 3 were used for association and monitored for 2 min, then 500 nM of V_{H} 115 were used for continuing association and monitored for 2 min. For affinity assay, 400 nM, 200 nM and 100 nM of V_{H}, V_{H}-Fc, and DbTE were used separately for association and monitored for 2 min. Dissociation was monitored in DPBS for 4 min.

Size exclusion chromatography: The aggregation of the antibodies was analyzed by Superdex 200 Increase 10/300 GL chromatography (GE Healthcare, Chicago, IL, USA) as previously described. 200 μg of filtered antibodies were analyzed and eluted by DPBS buffer at a flow rate of 0.5 mL/min.

Cells: Expi293 cells (Thermo Fisher) were maintained in an Expi293 expression medium supplemented with 0.4% penicillin-streptomycin (P/S). 293T cells and A375 human melanoma cells were purchased from ATCC and were maintained in DMEM medium supplemented with 10% FBS and 1% P/S separately. T cells were isolated from healthy donor's PBMCs (Zen-Bio) by using the human Pan T cell isolation kit (Miltenyi Biotec) and activated by CD3/CD28 T cell activator Dyna beads (Gibco) at 1:1 cell-bead ratio for 48 h. The activated T cells were used for the cytotoxicity assay of the DbTE antibody.

Flow Cytometry: The cell surface expression level of uPAR protein was detected by a commercial antibody. 2×10⁵ cells/test were stained with mouse anti-human uPAR antibody (R&D systems, Catalog #MAB807) or an isotype antibody for 30 min at 4° C. followed by PE-conjugated anti-mouse IgG secondary antibody. To verify the cell surface binding of the isolated antibody, cells were incubated with V_{H}-Fc 3 or V_{H}-Fc 115 at a concentration of 50 nM, or V_{H} 3 or V_{H} 115 at a concentration of 1 uM for 30 min at 4° C. Cells were then stained with a secondary antibody, goat anti-human IgG-PE (Sigma-Aldrich, 1:250) for V_{H}-Fc or anti-Flag-APC (Miltenyi Biotec) for V_{H}. An irrelevant V_{H}-Fc and V_{H} were used as isotype controls.

Cytotoxicity assays: The cell cytotoxicity of anti-uPAR DbTE was measured by LDH-Glo cytotoxicity assay kit (Promega) following the manufacturer's instructions. Target cells (1×10⁴ cells/well) and activated T cells were seeded in a 96-well plate at an E: T ratio 10:1, mixed with serially diluted DbTE antibodies in a growth medium, and incubated for 24 h at 37° C. in 5% CO₂ humidified atmosphere. The final volume was 100 μl/well. The cell supernatant was diluted 20-fold and incubated for 50 min for LDH assay setup. The calculation of relative % cytotoxicity is as follows: relative % cytotoxicity=100×(Experimental LDH release-Target and effector cell only)/(maximum LDH release control-Background).

Statistical Analysis: Statistical analyses were performed by GraphPad Prism. Differences were considered statistically significant when p<0.05. Significance was tested using two-way ANOVA, followed by Tukey's multiple comparisons tests. ****, p<0.0001.

### Results

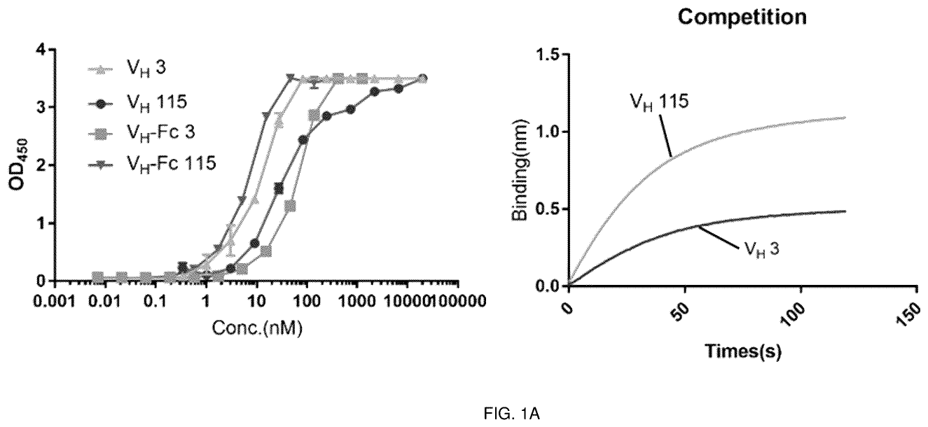

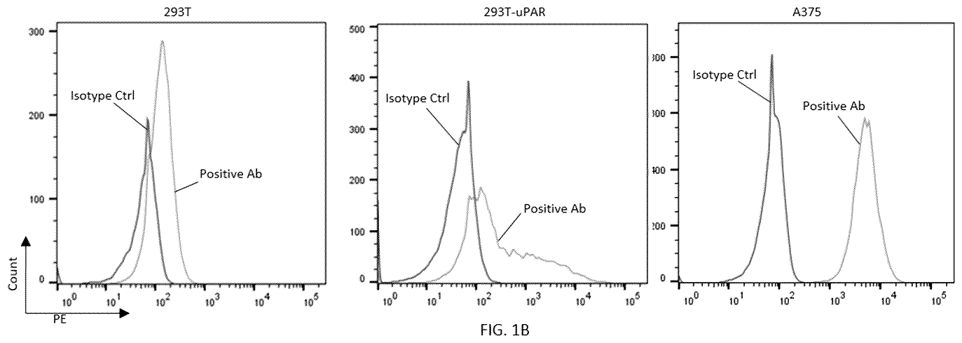

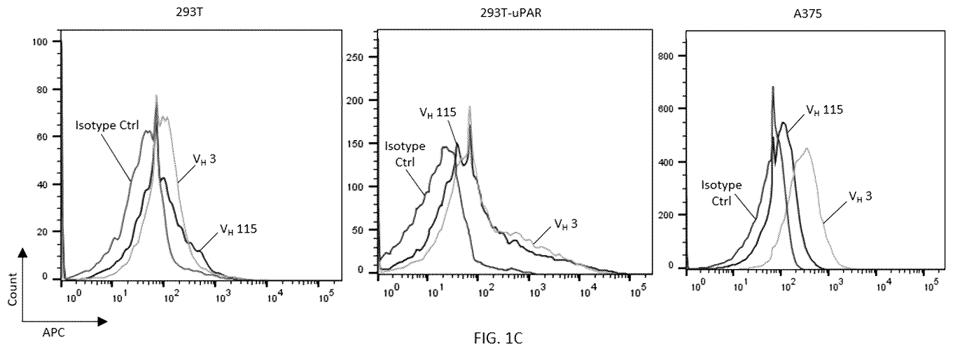

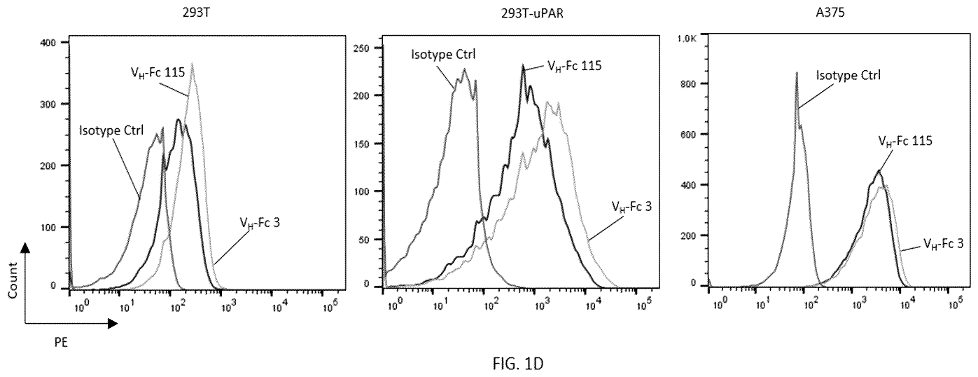

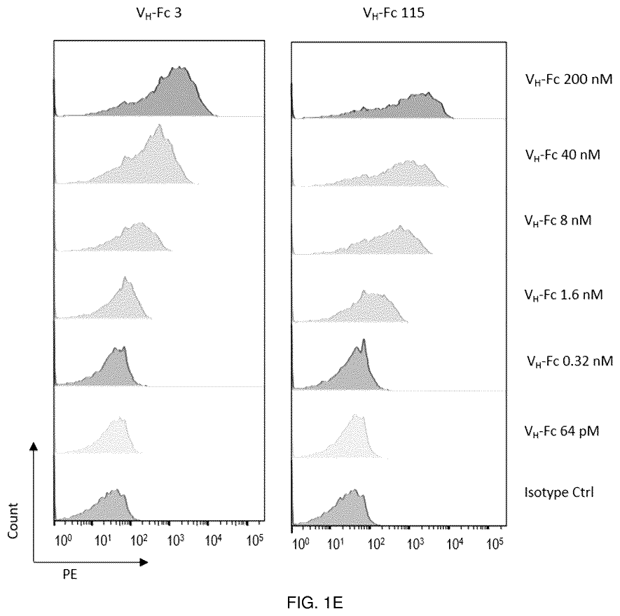

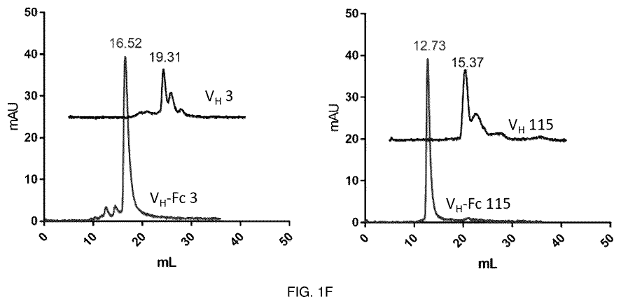

Selection and characterization of high-affinity V_{H} antibodies against human uPAR: A large phage-displayed human V_{H} library was used to pan against recombinant human uPAR protein for antibody selection. Several V_{H} binders were identified after three rounds of panning. Two antibodies, designated as V_{H} 3 and V_{H} 115, were selected based on their high affinity, specificity, and other desirable properties. The EC₅₀ values of V_{H} 3 and V_{H} 115 were 12.1+0.8 nM and 34.2+3.5 nM, respectively (FIG. 1A). The equilibrium dissociation constant (K_{D}) values were 17.1 nM and 1.7 nM respectively as determined by BLItz (Table 1). Additionally, the two binders did not bind to BSA at high concentrations, indicating their specificity for uPAR (data not shown). The competition BLItz experiment showed that V_{H} 3 and V_{H} 115 target different binding epitopes on human uPAR (FIG. 1A).

TABLE 1
BLItz results of human uPAR antibodies.

| Antibody | kₒₙ (M⁻¹s⁻¹)¹ | k_{off} (s⁻¹)¹ | K_{D} (nM)¹ |
|---|---|---|---|
| V_{H} 3 | 7.9 × 10⁴ ± 5.7 × 10² | 1.4 × 10⁻³ ± 1.9 × 10⁻⁵ | 17.1 |
| V_{H}-Fc 3 | 1.2 × 10⁵ ± 2.3 × 10³ | 1.2 × 10⁻³ ± 4.8 × 10⁻⁵ | 9.6 |
| DbTE 3 | 1.1 × 10⁵ ± 1.5 × 10³ | 7.2 × 10⁻⁴ ± 2.7 × 10⁻⁵ | 6.6 |
| V_{H} 115 | 2.1 × 10⁴ ± 2.9 × 10² | 3.6 × 10⁻⁵ ± 1.3 × 10⁻⁵ | 1.7 |
| V_{H}-Fc 115 | 3.3 × 10⁴ ± 1.5 × 10³ | 2.4 × 10⁻³ ± 5.6 × 10⁻⁵ | 71.1 |
| DbTE 115 | 3.6 × 10⁴ ± 4.1 × 10² | 4.2 × 10⁻⁵ ± 1.6 × 10⁻⁵ | 1.2 |

¹Mean kinetic rate constants (kₒₙ, k_{off}) and equilibrium dissociation constants (K_{D} = k_{off}/kₒₙ) were determined from curve fitting analyses of BLItz results.

To increase the binders' half-life and avidity, the two V_{H} binders were converted to V_{H}-Fc format by fusing IgG1 Fc into the C-terminal of V_{H}. The EC₅₀ values of V_{H}-Fc 3 and V_{H}-Fc 115 were 64.3+2.3 nM and 6.6+0.2 nM, respectively (FIG. 1A). The K_{D} values were 9.6 nM and 71.1 nM, respectively (Table 1). To verify the specificity of these binders to uPAR expressed on the cell surface, the surface expression of uPAR on parental 293T cells, 293T cells isogenically expressing uPAR (293T-uPAR), or A375 cells (human melanoma cell line intrinsically express uPAR) were verified by a commercial anti-human uPAR antibody. Among these cells, 293T cells (MFI of isotype vs positive uPAR Ab is 62 vs 126) showed a low expression of uPAR while 293T-uPAR cells (MFI of isotype vs positive uPAR Ab is 46.4 vs 176) and A375 cells (MFI of isotype vs positive uPAR Ab is 71.3 vs 4775) showed a high expression level of uPAR (FIGS. 1B-1D).

Next, the binding specificity of our newly identified binders was tested on the above cell lines. The two V_{H} and V_{H}-Fc binders showed a high binding to both 293T-uPAR and A375 cells, while a low level of binding to 293T cells (FIGS. 1B-1D). These results were consistent with the expression level of uPAR on these cell lines. Moreover, both V_{H}-Fc binders bound to the 293T-uPAR cells in a concentration-dependent manner (FIG. 1E). Protein folding was assessed by the size-exclusion chromatography (SEC). Based on the molecular weight calibration curves, while V_{H} 3 and V_{H}-Fc 3 exhibit monomeric folding, V_{H} 115 and V_{H}-Fc 115 both showed a dimeric folding (FIG. 1F). The late-elution peaks may be due to V_{H} interaction with the column agarose matrix. The stability of these V_{H} domains are further enhanced after converting to the V_{H}-Fc format. We found that the V_{H}-Fc proteins exhibit homogenous folding peaks. The Fc fragments may help to stabilize the V_{H} domain.

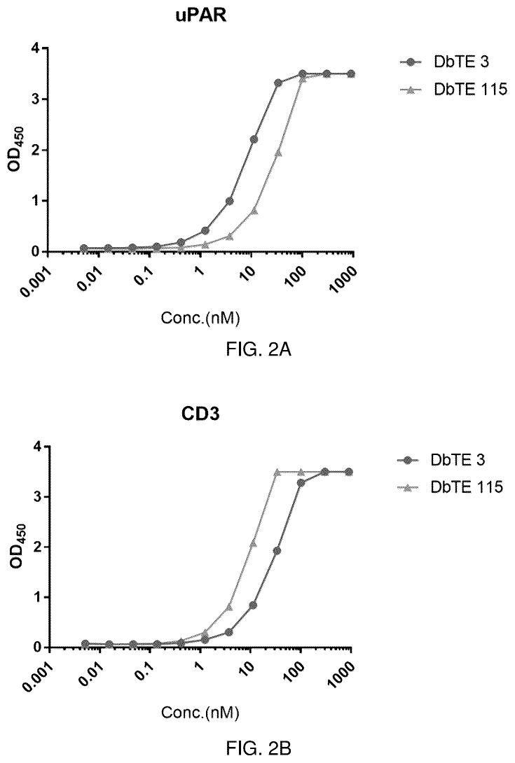

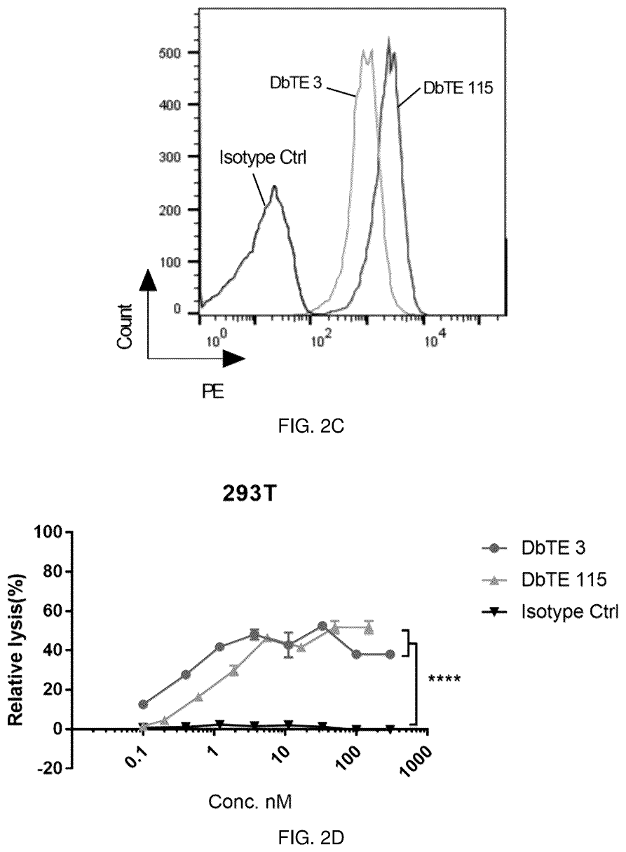

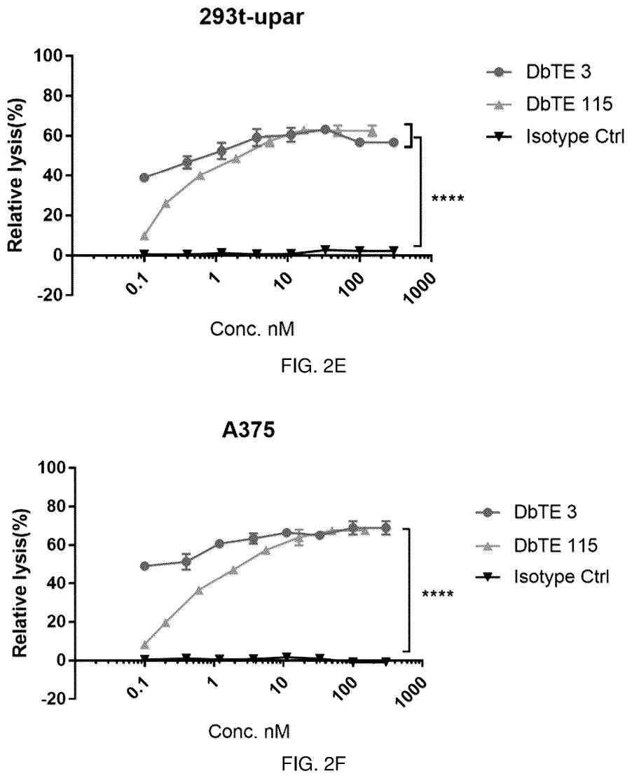

V_{H} domains-based T cell engagers (DbTEs) show potent cytotoxicity against uPAR expressing cells: As a proof of concept, we generated and assessed the cell cytotoxicity of anti-uPAR domain antibody-based bispecific T cell engagers (DbTEs) against uPAR expressing cancer cells. To construct DbTE, V_{H} domains were fused to the humanized anti-CD3 antibody OKT3 scFv, which is in frame to the human IgG1 Fc with FcγR binding silencing mutations (LALAPG (SEQ ID NO: 20)). The EC₅₀ of DbTE 3 and 115 for binding to the recombinant human uPAR protein as tested by ELISA were 7.7±0.4 nM and 28±2.1 nM, respectively (FIG. 2A). The EC₅₀ of DbTE 3 and 115 for binding to the human CD3 protein as tested by ELISA were 28.8±1.8 nM and 8.7±0.6 nM, respectively (FIG. 2B). DbTE binding to T cells was verified by flow cytometry (FIG. 2C).

Next, T-cell-mediated cytotoxicity against uPAR positive cancer cells induced by each DbTE was assessed using an LDH assay. Dose-dependent lysis was observed at the E: T ratio of 10:1 on 293T (FIG. 2D), 293T-uPAR cells (FIG. 2E), and A375 cells (FIG. 2F) mediated by DbTE 3 or DbTE 115. A lower level of lysis was observed with 293T cells, consistent its lower level of uPAR on the cell surface. Moreover, DbTE 3 appear to be more effective than DbTE 115 at low concentrations.

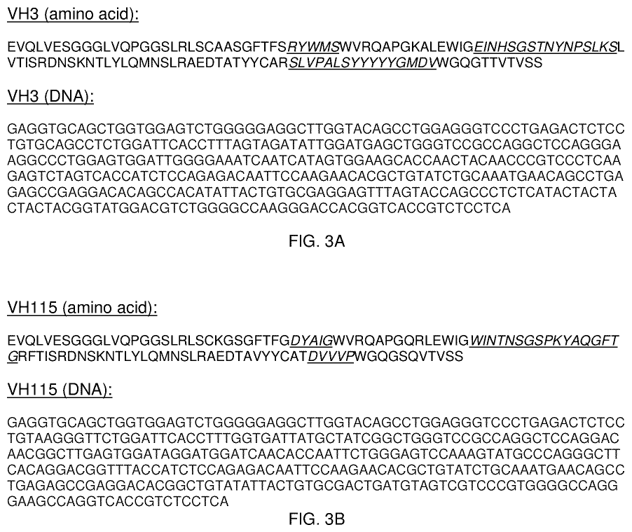

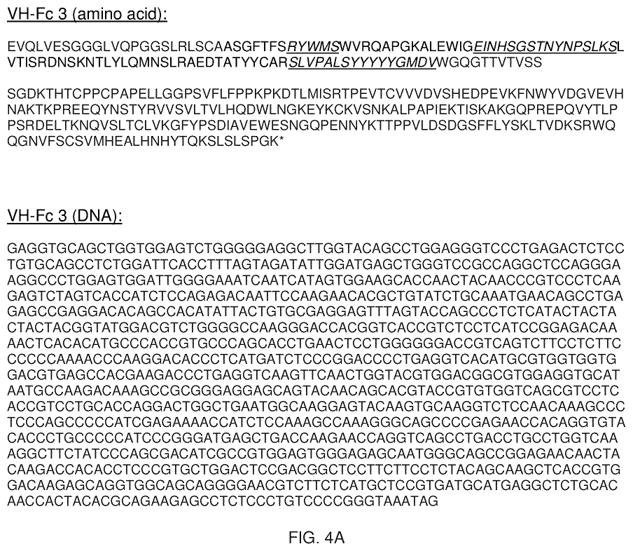

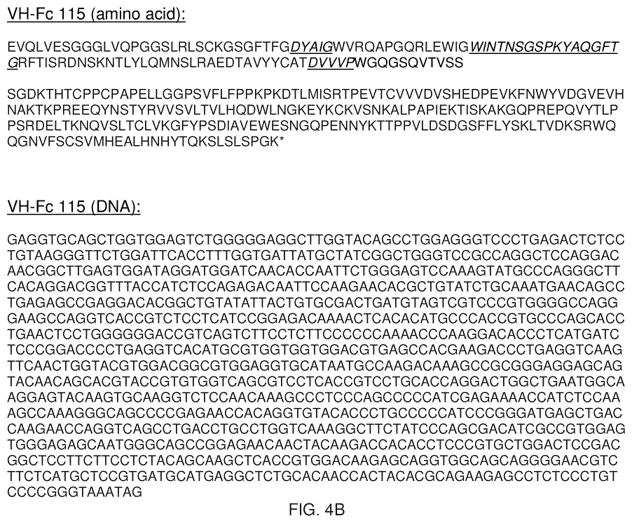

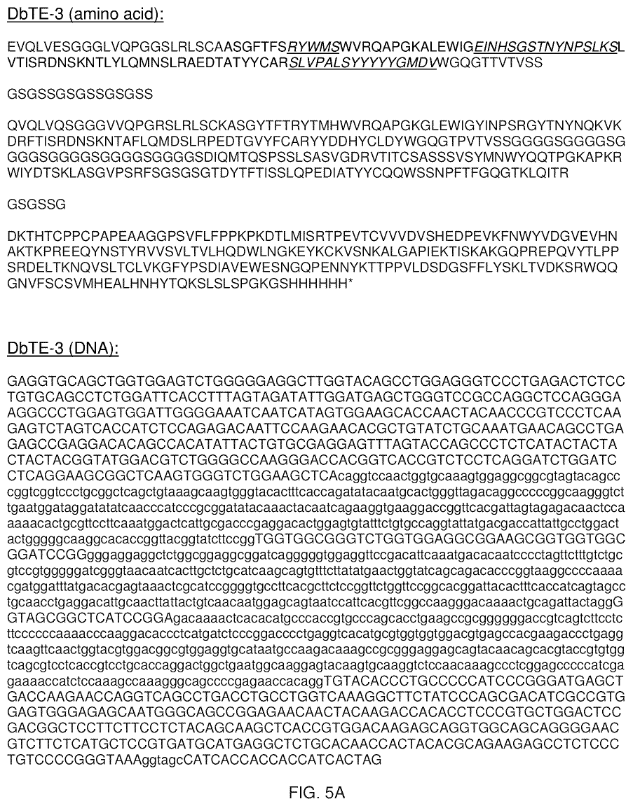

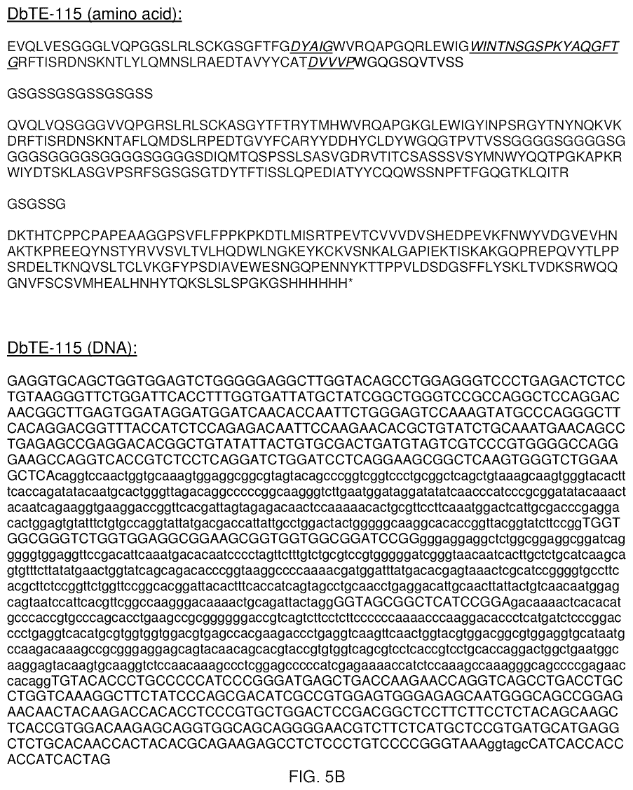

FIGS. 3A-5B provide exemplary amino acid sequences and exemplary corresponding DNA sequences of VH, VHFc, and DbTE compounds described in this example.

uPAR is a glycoprotein receptor that is highly expressed in many solid cancers including breast, lung, prostate, ovarian, and liver cancer. Moreover, uPAR is also highly expressed on stromal cells in the tumor microenvironment, such as vascular endothelial cells, tumor-related fibroblasts, and macrophages. The multifunctionality of uPAR ranging from tumor progression, invasion, and angiogenesis to metastasis makes it an ideal target for cancer therapy. Several antibody-based therapies targeting uPAR performed in preclinical showed promising effects in breast cancer but there have no antibody-based therapies targeting uPAR in clinical trials. However, several diagnostic clinical trials detecting uPAR for cancer and metastasis have demonstrate safe and clinical potential. Hence, characterization of novel antibodies with diverse affinity, specificity, and size may be useful in the treatment of cancers with high uPAR expression.

Two fully human V_{H} domain antibodies that target uPAR we selected and characterized. Both antibodies showed high affinity for uPAR. Converting the antibodies to V_{H}-Fc fusion protein enhanced the avidity of V_{H} 3 by 2-fold, but decreased avidity 40-fold in V_{H} 115 (FIG. 1A). This decrease may be due to the aggregation. Further antibody maturation of V_{H} 115 to decrease the aggregation may be employed. The killing effects of DbTEs based on these two antibodies showed specific cell killing of cells with observable expression levels of uPAR, demonstrating their potential for cancer immunotherapies. Lower killing effects were observed when targeting 293T cells with lower uPAR expression levels compared with the 293T-uPAR overexpressing cell line (FIGS. 2D and 2E). These findings support use of these reagents for cancer inhibition in vivo, and further supports the therapeutic potential of domain antibodies.

Recent studies have also shown that uPAR is associated with senescence-associated pathologies. As such, these antibodies may find use as senolytic reagents in senescence-related diseases, such as fibrotic disease, including fibrin-associated inflammation and liver fibrosis. In summary, the anti-uPAR antibodies described above showed significant potential in heavy chain variable domain antibody-based immunotherapies and may be useful in targeting diseases related to the elevated expression level of uPAR, including treatment of cancer and fibrotic diseases.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention. Thus, the invention is not limited by the description of the exemplary embodiments, but rather by the appended claims as originally filed.

1. An antigen binding molecule comprising a polypeptide comprising a uPAR-binding amino acid sequence, an antigen binding site, or CDRs of:
(SEQ ID NO: 1)
EVQLVESGGGLVQPGGSLRLSCAASGFTFSRYWMSWVRQAPGKALEW
IGEINHSGSTNYNPSLKSLVTISRDNSKNTLYLQMNSLRAEDTATYYCA
RSLVPALSYYYYYGMDVWGQGTTVTVSS;
(SEQ ID NO: 3)
EVQLVESGGGLVQPGGSLRLSCKGSGFTFGDYAIGWVRQAPGQRLEWI
GWINTNSGSPKYAQGFTGRFTISRDNSKNTLYLQMNSLRAEDTAVYYCA
TDVVVPWGQGSQVTVSS,
or a sequence having at least 70%, at least 75%, at least 80%, at least 90%, or at least 95% sequence identity with either of the preceding.

2. The antigen binding molecule of claim 1, comprising a uPAR-binding amino acid sequence having at least 70%, at least 75%, at least 80%, at least 90%, at least 95% or 100% sequence identity with:
(SEQ ID NO: 1)
EVQLVESGGGLVQPGGSLRLSCAASGFTFSRYWMSWVRQAPGKALEW
IGEINHSGSTNYNPSLKSLVTISRDNSKNTLYLQMNSLRAEDTATYYCA
RSLVPALSYYYYYGMDVWGQGTTVTVSS
and/or comprising the amino acid sequences:
VH3-HCDR1:
(SEQ ID NO: 13)
RYWMS;
VH3-HCDR2:
(SEQ ID NO: 14)
EINHSGSTNYNPSLKS;
and
VH3-HCDR3:
(SEQ ID NO: 15)
SLVPALSYYYYYGMDV.

3. The antigen binding molecule of claim 1, comprising a uPAR-binding amino acid sequence having at least 70%, at least 75%, at least 80%, at least 90%, at least 95% or 100% sequence identity with:
(SEQ ID NO: 3)
EVQLVESGGGLVQPGGSLRLSCKGSGFTFGDYAIGWVRQAPGQRLEWI
GWINTNSGSPKYAQGFTGRFTISRDNSKNTLYLQMNSLRAEDTAVYYCA
TDVVVPWGQGSQVTVSS
and/or comprising the amino acid sequences:
VH115-HCDR1:
(SEQ ID NO: 16)
DYAIG;
VH115-HCDR2:
(SEQ ID NO: 17)
WINTNSGSPKYAQGFTG;
and
VH115-HCDR3:
(SEQ ID NO: 18)
DVVVP.

4. The antigen binding molecule of claim 1, in the form of a nanobody comprising the uPAR-binding amino acid sequence.

5. The antigen binding molecule of claim 4:
having a sequence:
(SEQ ID NO: 1)
EVQLVESGGGLVQPGGSLRLSCAASGFTFSRYWMSWVRQAPG
KALEWIGEINHSGSTNYNPSLKSLVTISRDNSKNTLYLQMNSLRAEDTA
TYYCARSLVPALSYYYYYGMDVWGQGTTVTVSS;
having at least 70%, at least 75%, at least 80%, at least 90%, at least 95%, sequence identity with:
(SEQ ID NO: 1)
EVQLVESGGGLVQPGGSLRLSCAASGFTFSRYWMSWVRQAPG
KALEWIGEINHSGSTNYNPSLKSLVTISRDNSKNTLYLQMNSLRAEDTA
TYYCARSLVPALSYYYYYGMDVWGQGTTVTVSS;
having a sequence:
(SEQ ID NO: 3)
EVQLVESGGGLVQPGGSLRLSCKGSGFTFGDYAIGWVRQAPGQ
RLEWIGWINTNSGSPKYAQGFTGRFTISRDNSKNTLYLQMNSLRAEDTA
VYYCATDVVVPWGQGSQVTVSS;
or
having at least 70%, at least 75%, at least 80%, at least 90%, or at least 95%, sequence identity with:
(SEQ ID NO: 3)
EVQLVESGGGLVQPGGSLRLSCKGSGFTFGDYAIGWVRQAPGQ
RLEWIGWINTNSGSPKYAQGFTGRFTISRDNSKNTLYLQMNSLRAEDTA
VYYCATDVVVPWGQGSQVTVSS.

6. The antigen binding molecule of claim 1, in the form of a V_{H}-Fc fusion protein, comprising the uPAR-binding amino acid sequence.

7. The antigen binding molecule of claim 6:
having a sequence:
(SEQ ID NO: 5)
EVQLVESGGGLVQPGGSLRLSCAASGFTFSRYWMSWVRQAPG
KALEWIGEINHSGSTNYNPSLKSLVTISRDNSKNTLYLQMNSLRAEDTA
TYYCARSLVPALSYYYYYGMDVWGQGTTVTVSSSGDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHE
ALHNHYTQKSLSLSPGK;
having at least 70%, at least 75%, at least 80%, at least 90%, or at least 95% sequence identity with:
(SEQ ID NO: 5)
EVQLVESGGGLVQPGGSLRLSCAASGFTFSRYWMSWVRQAPG
KALEWIGEINHSGSTNYNPSLKSLVTISRDNSKNTLYLQMNSLRAEDTA
TYYCARSLVPALSYYYYYGMDVWGQGTTVTVSSSGDKTHTCPPCPAPEL
LGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHE
ALHNHYTQKSLSLSPGK;
having a sequence:
(SEQ ID NO: 7)
EVQLVESGGGLVQPGGSLRLSCKGSGFTFGDYAIGWVRQAPGQ
RLEWIGWINTNSGSPKYAQGFTGRFTISRDNSKNTLYLQMNSLRAEDTA
VYYCATDVVVPWGQGSQVTVSSSGDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSL
SLSPGK;
or
having at least 70%, at least 75%, at least 80%, at least 90%, at least 95%, or 100% sequence identity with:
(SEQ ID NO: 7)
EVQLVESGGGLVQPGGSLRLSCKGSGFTFGDYAIGWVRQAPGQ
RLEWIGWINTNSGSPKYAQGFTGRFTISRDNSKNTLYLQMNSLRAEDTA
VYYCATDVVVPWGQGSQVTVSSSGDKTHTCPPCPAPELLGGPSVFLFPP
KPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYK
TTPPVLDSDGSFFLYSKLTVDKSRWQQGNVFSCSVMHEALHNHYTQKSL
SLSPGK.

8. The antigen binding molecule of claim 1, in the form of a bi-specific T-cell engager, comprising the uPAR-binding amino acid sequence.

9. The antigen binding molecule of claim 8:
having a sequence:
(SEQ ID NO: 9)
EVQLVESGGGLVQPGGSLRLSCAASGFTFSRYWMSWVRQAPG
KALEWIGEINHSGSTNYNPSLKSLVTISRDNSKNTLYLQMNSLRAEDTA
TYYCARSLVPALSYYYYYGMDVWGQGTTVTVSSGSGSSGSGSSGSGSSQ
VQLVQSGGGVVQPGRSLRLSCKASGYTFTRYTMHWVRQAPGKGLEWIGY
INPSRGYTNYNQKVKDRFTISRDNSKNTAFLQMDSLRPEDTGVYFCARY
YDDHYCLDYWGQGTPVTVSSGGGGSGGGGGGGGSGGGGSGGGGSGGGGS
DIQMTQSPSSLSASVGDRVTITCSASSSVSYMNWYQQTPGKAPKRWIYD
TSKLASGVPSRFSGSGSGTDYTFTISSLQPEDIATYYCQQWSSNPFTFG
QGTKLQITRGSGSSGDKTHTCPPCPAPEAAGGPSVFLFPPKPKDTLMIS
RTPEVTCVVVDVSHEDPEVKFNWYVDGVEVHNAKTKPREEQYNSTYRVV
SVLTVLHQDWLNGKEYKCKVSNKALGAPIEKTISKAKGQPREPQVYTLP
PSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGKGSH
HHHHH;
having at least 70%, at least 75%, at least 80%, at least 90%, at least 95% or 100% sequence identity with:
(SEQ ID NO: 9)
EVQLVESGGGLVQPGGSLRLSCAASGFTFSRYWMSWVRQAPG
KALEWIGEINHSGSTNYNPSLKSLVTISRDNSKNTLYLQMNSLRAEDTA
TYYCARSLVPALSYYYYYGMDVWGQGTTVTVSSGSGSSGSGSSGSGSSQ
VQLVQSGGGVVQPGRSLRLSCKASGYTFTRYTMHWVRQAPGKGLEWIGY
INPSRGYTNYNQKVKDRFTISRDNSKNTAFLQMDSLRPEDTGVYFCARY
YDDHYCLDYWGQGTPVTVSSGGGGSGGGGSGGGGSGGGGSGGGGSGGGG
SDIQMTQSPSSLSASVGDRVTITCSASSSVSYMNWYQQTPGKAPKRWIY
DTSKLASGVPSRFSGSGSGTDYTFTISSLQPEDIATYYCQQWSSNPFTF
GQGTKLQITRGSGSSGDKTHTCPPCPAPEAAGGPSVFLFPPKPKDTLMI
SRTPEVTCVVVDVSHEDPEVKFNWYVDGVEVHNAKTKPREEQYNSTYRV
VSVLTVLHQDWLNGKEYKCKVSNKALGAPIEKTISKAKGQPREPQVYTL
PPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGKGS
HHHHHH;
having a sequence:
(SEQ ID NO: 11)
EVQLVESGGGLVQPGGSLRLSCKGSGFTFGDYAIGWVRQAPGQ
RLEWIGWINTNSGSPKYAQGFTGRFTISRDNSKNTLYLQMNSLRAEDTA
VYYCATDVVVPWGQGSQVTVSSGSGSSGSGSSGSGSSQVQLVQSGGGVV
QPGRSLRLSCKASGYTFTRYTMHWVRQAPGKGLEWIGYINPSRGYTNYN
QKVKDRFTISRDNSKNTAFLQMDSLRPEDTGVYFCARYYDDHYCLDYWG
QGTPVTVSSGGGGSGGGGSGGGGSGGGGSGGGGSGGGGSDIQMTQSPSS
LSASVGDRVTITCSASSSVSYMNWYQQTPGKAPKRWIYDTSKLASGVPS
RFSGSGSGTDYTFTISSLQPEDIATYYCQQWSSNPFTFGQGTKLQITRG
SGSSGDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKGSHHHHHH;
or
having at least 70%, at least 75%, at least 80%, at least 90%, at least 95% or 100% sequence identity with:
(SEQ ID NO: 11)
EVQLVESGGGLVQPGGSLRLSCKGSGFTFGDYAIGWVRQAPGQ
RLEWIGWINTNSGSPKYAQGFTGRFTISRDNSKNTLYLQMNSLRAEDTA
VYYCATDVVVPWGQGSQVTVSSGSGSSGSGSSGSGSSQVQLVQSGGGVV
QPGRSLRLSCKASGYTFTRYTMHWVRQAPGKGLEWIGYINPSRGYTNYN
QKVKDRFTISRDNSKNTAFLQMDSLRPEDTGVYFCARYYDDHYCLDYWG
QGTPVTVSSGGGGSGGGGSGGGGSGGGGSGGGGSGGGGSDIQMTQSPSS
LSASVGDRVTITCSASSSVSYMNWYQQTPGKAPKRWIYDTSKLASGVPS
RFSGSGSGTDYTFTISSLQPEDIATYYCQQWSSNPFTFGQGTKLQITRG
SGSSGDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALGAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGKGSHHHHHH.

10. The antigen binding molecule of claim 1, wherein the K_{D} for antigen binding of the uPAR-binding amino acid sequence to uPAR protein is 100 nM or less, as determined by surface plasmon resonance or biolayer interferometry (BLITZ).

11. A composition comprising the antigen binding molecule of claim 1 and a pharmaceutically-acceptable excipient.

12. The antigen binding molecule of claim 1, in the form of a chimeric antigen receptor, comprising the uPAR-binding amino acid sequence.

13. A T-cell or NK cell comprising the antigen binding molecule of claim 12.

14. An antibody drug conjugate (ADC) comprising an antibody binding molecule as claimed in claim 1 linked to a cytotoxic payload via a chemical linker.

15. The ADC of claim 14, wherein the cytotoxic payload is a microtubule-disrupting agent, a DNA-damaging agent, an RNA-targeting payload, an immune payload, a Bcl-xL inhibitor, a NAMPT inhibitor, or proteasome inhibitors, such as auristatin, a maytansinoid, an eribulin (e.g., eribulin mesylate), a tubulysins, a cryptophycins, an EG5 inhibitor, calicheamicin, a duocarmycin, doxorubicin, enediyne, a topoisomerase I inhibitor, a pyrrolo[2,1-c][1,4] benzodiazepine, a thailanstatins, an amatoxin, a Toll-like receptor agonist, a STING agonist, a glucocorticoid receptor modulator, or a carmaphycin, and/or the chemical linker is an acid-labile linker, a lysosomal protease-sensitive linker, a 3-glucuronide linker, a glutathione-sensitive disulfide linker, such as an ester-containing linker, a hydrazone-containing linker, a valine-citrulline (v-c)-containing linker, a valine-alanine (v-a)-containing linker, or a phenylalanine-lysine (p-I)-containing linker.

16. A composition comprising the ADC of claim 14 and a pharmaceutically-acceptable excipient.

17. An antigen binding reagent comprising a means for binding a uPAR protein on a surface of a cell comprising CDRs of VH3 or VH115.

18. A method of reducing a population of uPAR-expressing cells in a patient, comprising administering to the patient an amount of the composition of claim 11 effective to reduce the number of cells of the population of uPAR-expressing cells in the patient.

19. A method of treating a cancer, such as breast cancer, colorectal cancer, melanoma, brain cancer, lung cancer, ovarian cancer, prostate cancer, bladder cancer, liver cancer, gastric cancer, pancreatic cancer, a glioma, or a hematologic malignancy, such as acute myeloid leukemia or myeloma, in a patient, comprising administering to the patient an amount of the composition of claim 11 effective to treat the cancer in the patient.

20. A method of treating a fibrotic disease, such as systemic sclerosis (SSc), idiopathic pulmonary fibrosis, renal fibrosis, ureteral fibrosis, urethral fibrosis, bladder fibrosis, Peyronie's disease, liver cirrhosis, or COPD, or a senescence-related disease, such as fibrin-associated inflammation or liver fibrosis, in a patient, comprising administering to the patient an amount of the composition of claim 11 effective to treat the fibrotic disease in the patient.


SEQUENCE LISTING (20 sequences)

SEQ ID NO: 1 - protein, 124 aa, Homo sapiens
EVQLVESGGGLVQPGGSLRLSCAASGFTFSRYWMSWVRQAPGKALEWIGEINHSGSTNYNPSLKSLVTISRDNSKNTLYLQMNSLRAEDTATYYCARSLVPALSYYYYYGMDVWGQGTTVTVSS

SEQ ID NO: 2 - DNA, 372 bp, Homo sapiens
  source 1..372: mol_type = unassigned DNA
gaggtgcagctggtggagtctgggggaggcttggtacagcctggagggtccctgagactctcctgtgcagcctctggattcacctttagtagatattggatgagctgggtccgccaggctccagggaaggccctggagtggattggggaaatcaatcatagtggaagcaccaactacaacccgtccctcaagagtctagtcaccatctccagagacaattccaagaacacgctgtatctgcaaatgaacagcctgagagccgaggacacagccacatattactgtgcgaggagtttagtaccagccctctcatactactactactacggtatggacgtctggggccaagggaccacggtcaccgtctcctca

SEQ ID NO: 3 - protein, 114 aa, Homo sapiens
EVQLVESGGGLVQPGGSLRLSCKGSGFTFGDYAIGWVRQAPGQRLEWIGWINTNSGSPKYAQGFTGRFTISRDNSKNTLYLQMNSLRAEDTAVYYCATDVVVPWGQGSQVTVSS

SEQ ID NO: 4 - DNA, 342 bp, Homo sapiens
  source 1..342: mol_type = unassigned DNA
gaggtgcagctggtggagtctgggggaggcttggtacagcctggagggtccctgagactctcctgtaagggttctggattcacctttggtgattatgctatcggctgggtccgccaggctccaggacaacggcttgagtggataggatggatcaacaccaattctgggagtccaaagtatgcccagggcttcacaggacggtttaccatctccagagacaattccaagaacacgctgtatctgcaaatgaacagcctgagagccgaggacacggctgtatattactgtgcgactgatgtagtcgtcccgtggggccagggaagccaggtcaccgtctcctca

SEQ ID NO: 5 - protein, 353 aa, Homo sapiens
EVQLVESGGGLVQPGGSLRLSCAASGFTFSRYWMSWVRQAPGKALEWIGEINHSGSTNYNPSLKSLVTISRDNSKNTLYLQMNSLRAEDTATYYCARSLVPALSYYYYYGMDVWGQGTTVTVSSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1062 bp, Homo sapiens
  source 1..1062: mol_type = unassigned DNA
gaggtgcagctggtggagtctgggggaggcttggtacagcctggagggtccctgagactctcctgtgcagcctctggattcacctttagtagatattggatgagctgggtccgccaggctccagggaaggccctggagtggattggggaaatcaatcatagtggaagcaccaactacaacccgtccctcaagagtctagtcaccatctccagagacaattccaagaacacgctgtatctgcaaatgaacagcctgagagccgaggacacagccacatattactgtgcgaggagtttagtaccagccctctcatactactactactacggtatggacgtctggggccaagggaccacggtcaccgtctcctcatccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaatag

SEQ ID NO: 7 - protein, 343 aa, Homo sapiens
EVQLVESGGGLVQPGGSLRLSCKGSGFTFGDYAIGWVRQAPGQRLEWIGWINTNSGSPKYAQGFTGRFTISRDNSKNTLYLQMNSLRAEDTAVYYCATDVVVPWGQGSQVTVSSSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 1032 bp, Homo sapiens
  source 1..1032: mol_type = unassigned DNA
gaggtgcagctggtggagtctgggggaggcttggtacagcctggagggtccctgagactctcctgtaagggttctggattcacctttggtgattatgctatcggctgggtccgccaggctccaggacaacggcttgagtggataggatggatcaacaccaattctgggagtccaaagtatgcccagggcttcacaggacggtttaccatctccagagacaattccaagaacacgctgtatctgcaaatgaacagcctgagagccgaggacacggctgtatattactgtgcgactgatgtagtcgtcccgtggggccagggaagccaggtcaccgtctcctcatccggagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaatag

SEQ ID NO: 9 - protein, 636 aa, Homo sapiens
EVQLVESGGGLVQPGGSLRLSCAASGFTFSRYWMSWVRQAPGKALEWIGEINHSGSTNYNPSLKSLVTISRDNSKNTLYLQMNSLRAEDTATYYCARSLVPALSYYYYYGMDVWGQGTTVTVSSGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSHHHHHH

SEQ ID NO: 10 - DNA, 1911 bp, Homo sapiens
  source 1..1911: mol_type = unassigned DNA
gaggtgcagctggtggagtctgggggaggcttggtacagcctggagggtccctgagactctcctgtgcagcctctggattcacctttagtagatattggatgagctgggtccgccaggctccagggaaggccctggagtggattggggaaatcaatcatagtggaagcaccaactacaacccgtccctcaagagtctagtcaccatctccagagacaattccaagaacacgctgtatctgcaaatgaacagcctgagagccgaggacacagccacatattactgtgcgaggagtttagtaccagccctctcatactactactactacggtatggacgtctggggccaagggaccacggtcaccgtctcctcaggatctggatcctcaggaagcggctcaagtgggtctggaagctcacaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggtggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactaggggtagcggctcatccggagacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaaggtagccatcaccaccaccatcactag

SEQ ID NO: 11 - protein, 626 aa, Homo sapiens
EVQLVESGGGLVQPGGSLRLSCKGSGFTFGDYAIGWVRQAPGQRLEWIGWINTNSGSPKYAQGFTGRFTISRDNSKNTLYLQMNSLRAEDTAVYYCATDVVVPWGQGSQVTVSSGSGSSGSGSSGSGSSQVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSGGGGSGGGGSGGGGSGGGGSGGGGSGGGGSDIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRGSGSSGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSHHHHHH

SEQ ID NO: 12 - DNA, 1881 bp, Homo sapiens
  source 1..1881: mol_type = unassigned DNA
gaggtgcagctggtggagtctgggggaggcttggtacagcctggagggtccctgagactctcctgtaagggttctggattcacctttggtgattatgctatcggctgggtccgccaggctccaggacaacggcttgagtggataggatggatcaacaccaattctgggagtccaaagtatgcccagggcttcacaggacggtttaccatctccagagacaattccaagaacacgctgtatctgcaaatgaacagcctgagagccgaggacacggctgtatattactgtgcgactgatgtagtcgtcccgtggggccagggaagccaggtcaccgtctcctcaggatctggatcctcaggaagcggctcaagtgggtctggaagctcacaggtccaactggtgcaaagtggaggcggcgtagtacagcccggtcggtccctgcggctcagctgtaaagcaagtgggtacactttcaccagatatacaatgcactgggttagacaggcccccggcaagggtcttgaatggataggatatatcaacccatcccgcggatatacaaactacaatcagaaggtgaaggaccggttcacgattagtagagacaactccaaaaacactgcgttccttcaaatggactcattgcgacccgaggacactggagtgtatttctgtgccaggtattatgacgaccattattgcctggactactgggggcaaggcacaccggttacggtatcttccggtggtggcgggtctggtggaggcggaagcggtggtggcggatccgggggaggaggctctggcggaggcggatcagggggtggaggttccgacattcaaatgacacaatcccctagttctttgtctgcgtccgtgggggatcgggtaacaatcacttgctctgcatcaagcagtgtttcttatatgaactggtatcagcagacacccggtaaggccccaaaacgatggatttatgacacgagtaaactcgcatccggggtgccttcacgcttctccggttctggttccggcacggattacactttcaccatcagtagcctgcaacctgaggacattgcaacttattactgtcaacaatggagcagtaatccattcacgttcggccaagggacaaaactgcagattactaggggtagcggctcatccggagacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacacctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtccccgggtaaaggtagccatcaccaccaccatcactag

SEQ ID NO: 13 - protein, 5 aa, synthetic construct
RYWMS

SEQ ID NO: 14 - protein, 16 aa, synthetic construct
EINHSGSTNYNPSLKS

SEQ ID NO: 15 - protein, 16 aa, synthetic construct
SLVPALSYYYYYGMDV

SEQ ID NO: 16 - protein, 5 aa, synthetic construct
DYAIG

SEQ ID NO: 17 - protein, 17 aa, synthetic construct
WINTNSGSPKYAQGFTG

SEQ ID NO: 18 - protein, 5 aa, synthetic construct
DVVVP

SEQ ID NO: 19 - protein, 30 aa, synthetic construct
GGGGSGGGGSGGGGSGGGGSGGGGSGGGGS

SEQ ID NO: 20 - protein, 6 aa, synthetic construct
LALAPG